(19) United States
(12) Patent Application Publication
(10) Pub. No.: US 2010/0075942 A1
(43) Pub. Date: Mar. 25, 2010

(54) N-PHENYL-4-PYRIDIN-2-YL-BENZAMIDE DERIVATIVES AS HISTONE DEACYLASE (HDAC) INHIBITORS FOR THE TREATMENT OF CANCER

(75) Inventors: Keith Hopkinson Gibson, Macclesfield (GB); Elaine Sophie, Elizabeth Stokes, Macclesfield (GB); Michael James Waring, Macclesfield (GB); David Michael Andrews, Macclesfield (GB); Zbigniew Stanley Matusiak, Macclesfield (GB); Mark Maybury, Macclesfield (GB); Craig Anthony Roberts, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (unknown)

(21) Appl. No.: 11/813,838

(22) PCT Filed: Jan. 12, 2006
(86) PCT No.: PCT/GB06/00102

§ 371 Date: Jul. 12, 2007

(30) Foreign Application Priority Data
Jan. 15, 2005 (GB) 0500828.9

(51) Int. Cl. A61K 31/4418; C07D 213/61; C07D 401/12; C07D 213/82; C07D 401/06; A61K 31/496; A61K 31/4523

(52) U.S. Cl. 514/210.2; 546/337; 546/276.4; 546/268.1; 546/316; 544/360; 514/357; 514/355; 514/253.01

(57) ABSTRACT

The invention concerns benzamide derivatives of Formula (I) wherein R^{1a}, R^{1b}, R^{1c}, R², R³, R⁴, W, m and n have any of the meanings defined in the description; processes for their preparation, pharmaceutical compositions containing them and their use in the manufacture of a medicament for use as an antiproliferative agent in the prevention or treatment of tumours or other proliferative conditions which are sensitive to the inhibition of histone deacetylase (HDAC).

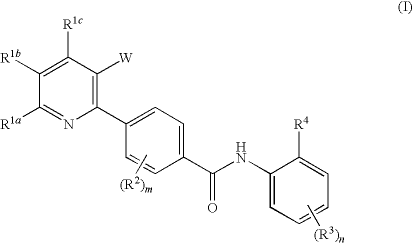

This invention relates to benzamide derivatives, or pharmaceutically acceptable salts or pro-drug forms thereof. The benzamide derivatives of the present invention are potent inhibitors of the enzyme histone deacetylase (HDAC), and are therefore useful agents for the treatment of disease states in which HDAC activity is known to be involved, such as cancer (Marks et al., Nature Reviews, 1, 194-202, (2001)), cystic fibrosis (Li, S. et al, J. Biol. Chem., 274, 7803-7815, (1999)), Huntingdons chorea (Steffan, J. S. et al., Nature, 413, 739-743, (2001)) and sickle cell anaemia (Gabbianelli, M. et al., Blood, 95, 3555-3561, (2000)). Accordingly, the present invention relates to methods for the treatment of any of the aforementioned conditions in a warm-blooded animal, such as man, by administering a pharmacologically active amount of a benzamide derivative of the present invention. The present invention also relates to processes for the manufacture of the benzamide derivatives of the present invention, to pharmaceutical compositions comprising these benzamide derivatives, and to their use of these derivatives in the manufacture of medicaments to inhibit HDAC in a warm-blooded animal, such as man.

In the eukaryotic cell, DNA is routinely compacted to prevent transcription factor accessibility. When the cell is activated this compacted DNA is made available to DNA-binding proteins, thereby allowing the induction of gene transcription (Beato, M., J. Med. Chem., 74, 711-724 (1996); Wolffe, A. P., Nature, 387, 16-17 (1997)). Nuclear DNA is known to associate with proteins known as histones to form a complex that is known as chromatin. The core histones, termed H2A, H2B, H3 and H4, are surrounded by 146 base pairs of DNA to form the fundamental unit of chromatin, which is known as the nucleosome. The N-terminal tails of the core histones contain lysine residues that are sites for post-transcriptional acetylation. Acetylation of the terminal amino group on the lysine side chain neutralizes the potential of the side chain to form a positive charge, and is thought to impact on chromatin structure.

Histone Deacetylases (HDACs) are zinc-containing enzymes which catalyse the removal of acetyl groups from the c-amino termini of lysine residues clustered near the amino terminus of nucleosomal histones. HDACs may be divided into two classes, the first (HDAC 1, 2, 3 and 8) represented by yeast Rpd3-like proteins, and the second (HDAC 4, 5, 6, 7, 9 and 10) represented by yeast Hda1-like proteins. The reversible process of acetylation is known to be important in transcriptional regulation and cell-cycle progression. In addition, HDAC deregulation has been associated with several cancers and HDAC inhibitors, such as Trichostatin A (a natural product isolated from Streptomyces hygroscopicus), have been shown to exhibit significant cell growth inhibition and anti-tumour effects (Meinke, P. T., Current Medicinal Chemistry, 8, 211-235 (2001)). Yoshida et al, (Exper. Cell Res., 177, 122-131 (1988)) teach that Trichostatin A causes the arrest of rat fibroblasts at the G1 and G2 phases of the cell cycle, thereby implicating the role of HDAC in the regulation of the cell cycle. Furthermore, Trichostatin A has been shown to induce terminal differentiation, inhibit cell growth, and prevent the formation of tumours in mice (Finnin et al., Nature, 401, 188-193 (1999)).

It is known from International Patent Publication Numbers WO 03/087057 and WO 03/092686, that certain benzamide derivatives are inhibitors of HDAC. One particular compound disclosed in WO 03/087057 is N-(2-aminophenyl)-4-pyridin-2-ylbenzamide.

However, there is no specific disclosure in either of these documents of benzamide derivatives which possess a further substituted-pyridin-2-yl group in the 4-position of the benzamide group. We have now found that certain benzamide derivatives possessing a substituted-pyridin-2-yl group in the 4-position are potent inhibitors of the HDAC enzyme.

According to the present invention there is provided a compound of formula (I) compound of formula (I):

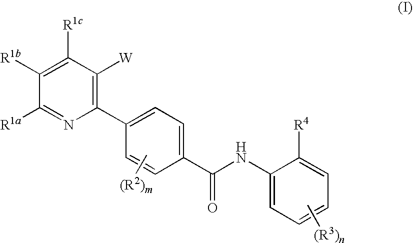

wherein:

R^{1a} is selected from hydrogen, amino, (1-3C)alkyl, N-(1-3C)alkylamino, N,N-di-(1-3C)alkylamino, or a group of the sub-formula II:

R⁵R⁶N—X¹—[CR^{a}R^{b}]_{q}— (II)

wherein:

q is 1, 2 or 3;

each R^{a} and R^{b} group present is independently selected from hydrogen, halo, hydroxy or (1-4C)alkyl;

X¹ is selected from a direct bond or —C(O)—; and
R⁵ and R⁶ are each independently selected from hydrogen or (1-3C)alkyl;

and wherein if R^{1a} is a N-(1-3C)alkylamino or N,N-di-(1-3C)alkylamino group, the (1-3C)alkyl moiety is optionally substituted by hydroxy or (1-2C)alkoxy;

R^{1b} is selected from:
(i) hydrogen, (1-6C)alkyl, halo(1-6C)alkyl, hydroxy(1-6C)alkyl, (3-6C)cycloalkyl, (3-6C)cycloalkyl(1-6C)alkyl, (1-6C)alkoxy, (1-6C)alkoxy(1-6C)alkyl, N-(1-6C)alkylsulphamoyl, N,N-di-[(1-6C)alkyl]sulphamoyl; or
(ii) a group of sub-formula III:

R⁷R⁸N—[CR^{a}R^{b}]ₐ—X²— (III)

wherein:
X² is selected from a direct bond, —O— or —C(O)—;

a is 0, 1, 2, 3 or 4;

R^{a} and R^{b} are as defined above;

R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IV:

R⁹R¹⁰N—[CR^{a}R^{b}]_{b}—X⁴— (IV)

wherein:

b is 1, 2 or 3;

R^{a} and R^{b} are as defined above;

X⁴ is a direct bond or —C(O)—;

R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by one or more hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cyclo alkyl(1-6C)alkyl);

or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S, or a group —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl); or
(iii) a group of the formula V:

R¹⁵R¹⁶N—X³—[CR^{a}R^{b}]_{c}— (V)

wherein:

c is 0, 1, 2 or 3;

R^{a} and R^{b} are as defined above;
X³ is —C(O)—;

R¹⁵ and R¹⁶ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula VI:

R¹⁷R¹⁸N—[CR^{a}R^{b}]_{d}— (VI)

wherein:

d is 1, 2 or 3;

R^{a} and R^{b} are as defined above;

R¹⁷ and R¹⁸ are independently selected from hydrogen, (1-6C)alkyl, (3-6C)cycloalkyl, (3-6C)cycloalkyl(1-6C)alkyl, (1-6C)alkoxy(1-6C)alkyl, or R¹⁷ and R¹⁸ are linked so that, together with the nitrogen atom to which they are attached, they form a 4-, 5-, 6- or 7-membered heterocyclic ring, said heterocyclic ring optionally comprising, in addition to the nitrogen atom to which R¹⁷ and R¹⁸ are attached, one or two further nitrogen atoms, and wherein the heterocyclic ring is optionally substituted by 1, 2 or 3, substituents selected from hydroxy, halo, (1-4C)alkyl, carbamoyl, oxo, or —[CH₂]_{g}—NR¹⁹R²⁰ (wherein g is 0, 1 or 2, and R¹⁹ and R²⁰ are independently selected from hydrogen, (1-6C)alkyl, (3-6C)cycloallcyl or (3-6C)cyclo alkyl(1-6C)alkyl);

or R¹⁵ and R¹⁶ are linked so that, together with the nitrogen atom to which they are attached, they form a 4-, 5-, 6- or 7-membered heterocyclic ring, said heterocyclic ring optionally comprising, in addition to the nitrogen atom to which R¹⁵ and R¹⁶ are attached, one or two further nitrogen atoms and the heterocyclic ring is optionally substituted by 1, 2 or 3, substituents selected from hydroxy, halo, (1-4C)alkyl, carbamoyl, oxo, or —[CH₂]ₕ—NR²¹R²² (wherein h is 0, 1 or 2, and R²¹ and R²² are independently selected from hydrogen, (1-6C)alkyl, (3-6C)cyclo alkyl or (3-6C)cyclo alkyl(1-6C) alkyl); or
(iv) a group of the sub-formula VII:

Q-Z—Y— (VII)

wherein:

Y is a direct bond or —[CR^{a}R^{b}]ₓ—, where x is 1 to 4 and R^{a} and R^{b} are as defined above;

Z is absent or selected from —O—, —S—, —SO—, —SO₂—, —NH—SO₂—,

—SO₂—NH— or —C(O)—; and

Q is a carbon-linked heterocyclyl or a heterocyclyl-(1-6C)alkyl group, said heterocyclyl or a heterocyclyl-(1-6C)alkyl group being optionally substituted on the heterocyclyl ring by one or more substituent groups (for example 1, 2 or 3), which may be the same or different, selected from halo, oxo, cyano, hydroxy, trifluoromethyl, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, (1-3C)alkoxy(1-3C)alkyl, (1-3C)alkoxycarbonyl, halo(1-3C)alkyl, N-[(1-3C)alkyl]amino, N,N-di-[(1-3C)alkyl]amino, N-[(1-3C)alkoxy(1-3C)alkyl]amino, N,N-di-[(1-3C)alkoxy(1-3C)alkyl]amino, N-[(1-3C)alkoxy(1-3C)alkyl]—N-[(1-3C)alkyl]amino, N-(1-3C)alkylcarbamoyl, N,N-di-[(1-3C)alkyl]carbamoyl, (1-3C)alkylthio, (1-3C)alkylsulphinyl, (1-3C)alkylsulphonyl, N-(1-3C)alkylsulphamoyl, N,N-di-[(1-3C)alkyl]sulphamoyl;

R^{1c} is selected from hydrogen, halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;
m is 0, 1, 2, 3 or 4;

R² is halo;

n is 0, 1, 2, 3 or 4;

R³ is selected from halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-Di(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;
R⁴ is amino or hydroxy; and
W is fluoro, chloro or bromo;
or a pharmaceutically acceptable salt or pro-drug thereof

It is to be understood that, insofar as certain of the compounds of Formula I defined above may exist in optically active or racemic forms by virtue of one or more asymmetric carbon atoms, the invention includes in its definition any such optically active or racemic fond which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect heterocyclic groups within the R^{1b} groups that bear 1 or 2 oxo substituents. It is to be understood that the present invention includes in its definition any such tautomeric form, or a mixture thereof, which possesses the above-mentioned activity and is not to be limited merely to any one tautomeric form utilised within the formulae drawings or named in the Examples.

Where optional substituents are selected from “one or more” substituent groups it is to be understood that this definition includes all substituents being chosen from one of the specified groups or the substituents being chosen from two or more of the specified groups.

In this specification the generic term “(1-6C)alkyl” includes both straight-chain and branched-chain alkyl groups such as propyl, isopropyl and tert-butyl, and also (3-6C)cycloalkyl groups such as cyclopropyl, cyclobutyl, cyclopentyl, and cyclohexyl, and also cycloalkyl-alkyl groups having 4 to 6 carbon atoms, such as cyclopropylmethyl, 2-cyclopropylethyl, cyclobutylmethyl, 2-cyclobutylethyl, and cyclopentylmethyl. However, references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only.

An analogous convention applies to other generic terms, for example (1-6C)alkoxy includes (3-6C)cycloalkyloxy groups and cycloalkyl-alkoxy groups having 4 to 6 carbon atoms, for example methoxy, ethoxy, propoxy, isopropoxy, cyclopropyloxy, cyclobutyloxy, cyclopentyloxy, cyclohexyloxy, cyclopropylmethoxy, 2-cyclopropylethoxy, cyclobutylmethoxy, 2-cyclobutylethoxy and cyclopentylmethoxy.

A person skilled in the art will appreciate that the terms “(1-4C)alkyl”, “(1-3C)alkyl” and “(1-2C)alkyl” are used herein refer to any of the alkyl groups defined above that posses 1 to 4, 1 to 3 and 1 to 2 carbon atoms respectively. The same convention applies to other terms used herein, such as, for example, “(1-4C)alkoxy”, “(1-3C)alkoxy” and “(1-2C)alkoxy”.

The term “halo” refers to fluoro, chloro, bromo and iodo.

Unless otherwise defined herein, the term “heterocyclyl” refers to a saturated, partially saturated or unsaturated, mono or bicyclic ring containing 3-12 atoms of which at least one atom is chosen from nitrogen, sulphur or oxygen, and which may, unless otherwise specified, be carbon or nitrogen linked, and wherein a CH₂ group can optionally be replaced by a C(O), and wherein a ring sulphur atom may be optionally oxidised to form the S-oxide(s). Preferably a “heterocyclyl” is a saturated, partially saturated or unsaturated, monocyclic ring containing 4, 5, 6 or 7 atoms, wherein at least one atom of the ring is chosen from nitrogen, sulphur or oxygen, and the ring system may, unless otherwise specified, be carbon or nitrogen linked, and wherein a ring sulphur atom may be optionally oxidised to form S-oxide(s). Examples and suitable values of the term “heterocyclyl” are aziridinyl, azetidinyl, thiazolidinyl, pyrrolidinyl, 1,3-benzodioxolyl, 1,2,4-oxadiazolyl, morpholinyl, tetrahydrofuranyl, furanyl, tetrahydropyranyl, piperidinyl, piperazinyl, thiomorpholinyl, 1,3-dioxolanyl, homopiperidinyl, homopiperazinyl, thienyl, pyrrolyl, pyrazolyl, oxadiazolyl, tetrazolyl, oxazolyl, thienopyrimidinyl, thienopyridinyl, thieno[3,2d]pyrimidinyl, 1,3,5-triazinyl, isoxazolyl, imidazolyl, thiadiazolyl, isothiazolyl, 1,2,3-triazolyl, 1,2,4-triazolyl, pyranyl, pyrimidyl, thiazolyl, pyridazinyl, and pyridyl.

Particular examples of 4-, 5- or 6-membered monocyclic heterocyclyl groups include azetidinyl, pyrrolidinyl, piperidinyl, piperazinyl, morpholinyl, pyrrolyl and pyridyl, and especially azetidin-1-yl, pyrrolidin-1-yl, piperidin-1-yl, piperazin-1-yl, morpholin-4-yl, pyrrol-1-yl, pyrid-1-yl, pyrid-2-yl, pyrid-3-yl and pyrid-4-yl.

Where a heterocyclyl group includes one or more nitrogen atoms, these may carry a hydrogen atom or a substituent group such as a (1-6C)alkyl group if required to fulfil the bonding requirements of nitrogen, or they may be linked to the rest of the structure by way of the nitrogen atom. A nitrogen atom within a heterocyclyl group may be oxidized to give the corresponding N oxide.

Within this specification composite terms are used to describe groups comprising more that one functionality, such as heterocyclyl-(1-6C)alkyl. These composite terms are to be given their ordinary meanings and will be understood by a person skilled in the art. For example, the term heterocyclyl(1-6C)alkyl refer to substituent groups wherein a heterocyclyl moiety is linked via a (1-6C)alkyl chain. The same convention also applies to other composite terms used herein, such as (1-6C)alkoxy(1-6C)alkyl and (3-6C)cycloalkyl(1-6C)alkyl.

Suitable values for groups in the definitions of any of R^{1a}, R^{1c}, or R³ are as follows:
for (1-3C)alkyl: methyl, ethyl, propyl, and isopropyl;
for (2-3C)alkenyl: vinyl, isopropenyl, and allyl;
for (2-3C)alkynyl: ethynyl, and 2-propynyl;
for (1-3C)alkanoyl: acetyl and propionyl;
for (1-3C)alkanoyloxy: acetoxy and propionyloxy;
for (1-3C)alkoxy: methoxy, ethoxy, and propoxy;
for N-(1-3C)alkylamino: methylamino, ethylamino, propylamino, and isopropylamino;
for N,N-di-[(1-3C)alkyl]amino: dimethylamino, diethylamino, N-ethyl-N-methylamino and diisopropylamino;
for (1-3C)alkanoylamino: acetamido and propionamido;
for N-(1-3C)alkylcarbamoyl: N-methylcarbamoyl, N-ethylcarbamoyl and N-propylcarbamoyl;
for N,N-di-(1-3C)alkylcarbamoyl: N,N-dimethylcarbamoyl, N-ethyl-N-methylcarbamoyl and N,N-diethylcarbamoyl;
for (1-3C)alkylthio: methylthio, ethylthio and propylthio;
for (1-3C)alkylsulphinyl: methylsulphinyl and ethylsulphinyl;
for (1-3C)alkylsulphonyl: methylsulphonyl and ethylsulphonyl;
for (1-3C)alkoxycarbonyl: methoxycarbonyl, ethoxycarbonyl, and propoxycarbonyl;
for N-(1-3C)alkylsulphamoyl: N-methylsulphamoyl and N-ethylsulphamoyl; and
for N,N-di-(1-3C)alkylsulphamoyl: N,N-dimethylsulphamoyl.

Suitable values for R^{1a} when it is a group of sub-formula II:

R⁵R⁶N—X¹—[CR^{a}R^{b}]_{q}—

include (methylamino)methyl, (ethylamino)methyl, (propylamino)methyl, (isopropylamino)methyl, (cyclopropylamino)methyl, (dimethylamino)methyl, N-methylcarbamoyl, N-ethylcarbamoyl, N-propylcarbamoyl; N,N-dimethylcarbamoyl, N-ethyl-N-methylcarbamoyl and N,N-diethylcarbamoyl.

Suitable values for R^{1b}, or moieties within a R^{1b} substituent group, are as follows:
for halo: fluoro, chloro, bromo and iodo;
for (1-6C)alkyl: methyl, ethyl, propyl, isopropyl, tert-butyl, cyclobutyl, cyclohexyl, cyclopropylmethyl, cyclobutylmethyl and 2-cyclopropylethyl;
for (1-4C)alkyl: methyl, ethyl, propyl, isopropyl, tert-butyl, and cyclobutyl;
for (1-2C)alkyl: methyl and ethyl;
for (2-3C)alkenyl: vinyl, isopropenyl, and allyl;
for (2-3C)alkynyl: ethynyl, and 2-propynyl;
for (3-6C)cycloalkyl: cyclopropyl, cyclobutyl, cyclopentyl, and cyclohexyl;
for (3-6C)cycloalkyl-(1-6C)alkyl: cyclopropylmethyl, cyclobutylmethyl, cyclopentyl methyl, 2-cyclopropylethyl and 2-cyclobutylpropyl;
for (1-6C)alkoxy: methoxy, ethoxy, propoxy, isopropoxy and butoxy;
for (1-6C)alkoxy(1-6C)alkyl: methoxymethyl, 2-methoxyethyl, 1-methoxyethyl, and 3-methoxypropyl;
for (1-3C)alkanoyl: acetyl and propionyl;
for (1-3C)alkanoyloxy: acetoxy and propionyloxy;
for (1-3C)alkoxycarbonyl: methoxycarbonyl and ethoxycarbonyl:
for halo(1-6C)alkyl: chloromethyl, 2-fluoroethyl, 2-chloroethyl, 1-chloroethyl, 2,2-difluoroethyl, 2,2,2-trifluoroethyl, 3-fluoropropyl, 3-chloropropyl, 3,3-difluoropropyl, 3,3,3-trifluoropropyl, and trifluoromethyl;
for hydroxy(1-6C)alkyl: hydroxymethyl, 2-hydroxyethyl, 1-hydroxyethyl and 3-hydroxypropyl;
for N-(1-3C)alkylamino: methylamino, ethylamino, propylamino, and isopropylamino;
for N,N-di-[(1-3C)alkyl]amino: dimethylamino, diethylamino, N-ethyl-N-methylamino and diisopropylamino;
for N-[(1-3C)alkoxy(1-3C)alkyl]amino: methoxymethylamino and 2-methoxyethylamino;
for N,N-di-[(1-3C)alkoxy(1-3C)alkyl]amino: di-methoxymethylamino and di-(2-methoxyethyl)amino;
for N-[(1-3C)alkoxy(1-3C)alkyl]—N-[(1-3C)alkyl]amino: N-(2-methoxyethyl)-N-methylamino, N-(2-methoxyethyl)-N-ethylamino and N-(2-methoxyethyl)-N-isopropylamino;
for (1-3C) alkylthio: methylthio, ethylthio and propylthio;
for (1-3C) alkylsulphinyl: methylsulphinyl and ethylsulphinyl;
for (1-3C) alkylsulphonyl: methylsulphonyl and ethylsulphonyl;
for (1-3C)alkoxycarbonyl: methoxycarbonyl, ethoxycarbonyl, and propoxycarbonyl;
for N-(1-3C)alkylcarbamoyl: N-methylcarbamoyl, N-ethylcarbamoyl and N-propylcarbamoyl;
for N,N-di-[(1-3C)alkyl]carbamoyl: N,N-dimethylcarbamoyl, N-ethyl-N-methylcarbamoyl and N,N-diethylcarbamoyl;
for N-(1-3C)alkylsulphamoyl: N-methylsulphamoyl and N-ethylsulphamoyl;
for N,N-di-[(1-3C)alkyl]sulphamoyl: N,N-dimethylsulphamoyl.

Examples of R^{1b} when it is a group of the sub-formula III:

R⁷R⁸N—[CR^{a}R^{b}]ₐ—X²—

include (methylamino)methyl, (ethylamino)methyl, (propylamino)methyl, (isopropylamino)methyl, (cyclopropylamino)methyl, (butylamino)methyl, (cyclobutylamino)methyl, (cyclopentylamino)methyl, (1-methylpropylamino)methyl, (2-methylpropylamino)methyl, 1-(methylamino)ethyl, 2-(methylamino)ethyl, 2-(ethylamino)ethyl, 3-(methylamino)propyl, (di-methylamino)methyl, (di-ethylamino)methyl, [(ethyl)(methyl)amino]methyl, [(isopropyl)(methyl)amino]methyl, [(propyl)(methyl)amino]methyl, [(cyclopropylmethyl)amino]methyl, [(cyclobutylmethyl)(methyl)amino]methyl, [(2-methoxyethyl)(methyl)amino]methyl, [(isopropyl)(2-methoxyethyl)amino]methyl, [(2-methoxyethyl)amino]methyl, [(ethyl)(2-methoxyethyl)amino]methyl, {[2-(di-methylamino)ethyl]amino}methyl, {[2-(di-ethylamino)ethyl]amino}methyl, {[2-(di-methylamino)ethyl][methyl]amino}methyl, {[2-(di-ethylamino)ethyl][methyl]amino}methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4-ethylpiperazin-1-yl)methyl, (4-isopropylpiperazin-1-yl)methyl, 2-(dimethylamino)ethoxy, 2-pyrrolidin-1-ylethoxy, and 2-(4-methylpiperazin-1-yl)ethoxy as well as N-2-[dimethylamino]ethyl-N-methyl-carbamoyl, N-2-[pyrrolidin-1-yl]ethyl-carbamoyl, N-2-[diethylaminolethyl-carbamoyl, (3-methoxypropyl)amino]methyl, [(3-ethoxypropyl)amino]methyl, [(2-ethoxyethyl)amino]methyl, [3-(methylsulfonyl)pyrrolidin-1-yl]methyl, [4-(2-methoxyethyl)piperazin-1-yl]methyl, and [(2-propoxyethyl)amino]methyl.

Suitably, when R^{1b} is a group of sub-formula III, it is selected from (methylamino)methyl, (ethylamino)methyl, (propylamino)methyl, (isopropylamino)methyl, (butylamino)methyl, (cyclobutylamino)methyl, (2-methylpropylamino)methyl, [(cyclopropylmethyl)amino]methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4-ethylpiperazin-1-yl)methyl, (4-isopropylpiperazin-1-yl)methyl, 2-(dimethylamino)ethoxy, 2-pyrrolidin-1-ylethoxy, and 2-(4-methylpiperazin-1-yl)ethoxy as well as N-2-[dimethylamino]ethyl-N-methyl-carbamoyl, N-2-[pyrrolidin-1-yl]ethyl-carbamoyl, N-2-[diethylamino]ethyl-carbamoyl, (3-methoxypropyl)amino]methyl, [(3-ethoxypropyl)amino]methyl, [(2-ethoxyethyl)amino]methyl, [3-(methylsulfonyl)pyrrolidin-1-yl]methyl, [4-(2-methoxyethyl)piperazin-1-yl]methyl, and [(2-propoxyethyl)amino]methyl.

Particular examples of R^{1b} when it is a group of the sub-formula V:

R¹⁵R¹⁶N—X³—[CR^{a}R^{b}]_{c}—

include N-[2-(diethylamino)ethyl]carbamoyl, N-(2-pyrrolidin-1-ylethyl)carbamoyl, and N-[2-(dimethylamino)ethyl)]-N-(methyl)carbamoyl.

Suitable examples of Q (sub-formula VII) when it is a heterocyclyl(1-6C)alkyl group include azetidinylmethyl, pyrrolidinylmethyl, 1-methyl-pyrrolidin-2-ylmethyl, piperidinylmethyl, piperazinylmethyl, morpholinylmethyl, 1-azetidinylethyl, 1-pyrrolidinylethyl, 1-piperidinylethyl, 1-piperazinylethyl, 1-morpholinylethyl, particularly 1-methyl-pyrrolidin-2-ylmethyl.

When Q is a heterocyclyl-(1-6C)alkyl group it is preferable that it is a heterocyclyl-(1-4C)alkyl group, and more preferably a heterocyclyl-(1-2C)alkyl, and especially a heterocyclyl-methyl group.

A particular example of group R^{1b} when it is a group of sub-formula VII:

Q-Z—Y—

is (1-methylpyrrolidin-2-yl)methoxy.

In an embodiment of the invention, it is also preferred that any heterocyclyl ring present within a R^{1b} substituent group contains nitrogen as the only heteroatom present. Suitably the heterocyclyl moiety contains one or two ring nitrogen atoms as the only heteroatoms present.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulphuric, trifluoroacetic, citric or maleic acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine. A further suitable pharmaceutically-acceptable salt of a compound of the Formula I is, for example, a salt formed within the human or animal body after administration of a compound of the Formula I.

The compounds of the invention may be administered in the foini of a pro-drug (that is a compound that is broken down in the human or animal body to release a compound of the invention). A pro-drug may be used to alter the physical properties and/or the phatinacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in vivo cleavable amide derivatives that may be formed at a carboxy group or an amino group in a compound of the Formula I.

Accordingly, the present invention includes those compounds of the Formula I as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I may be a synthetically-produced compound or a metabolically-produced compound.

A suitable pharmaceutically-acceptable pro-drug of a compound of the Formula I is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic

Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula I that possesses a carboxy group is, for example, an in vivo cleavable ester thereof. An in vivo cleavable ester of a compound of the Formula I containing a carboxy group is, for example, a pharmaceutically-acceptable ester which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I that possesses a hydroxy group is, for example, an in vivo cleavable ester or ether thereof. An in vivo cleavable ester or ether of a compound of the Formula I containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula I that possesses a carboxy group is, for example, an in vivo cleavable amide thereof, for example an 5 amide formed with an amine such as ammonia, a (1-4C)alkylamine such as methylamine, a di-(1-4C)alkylamine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically-acceptable pro-drug of a compound of the Formula I that possesses an amino group is, for example, an in vivo cleavable amide derivative thereof. Suitable pharmaceutically-acceptable amides from an amino group include, for example an amide formed with (1-10C)allcanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I may be exerted in part by one or more metabolites that are formed within the human or animal body after administration of a compound of the Formula I. As stated hereinbefore, the in vivo effects of a compound of the Formula I may also be exerted by way of metabolism of a precursor compound (a pro-drug).

In a particular group of compounds of the invention, when R^{1b} is a group of sub-formula (III) and where at least one of R⁷ or R⁸ is a group of sub-formula (IV), then X⁴ is a direct bond such that the group of sub-formula (IV) is a group of sub-formula (IVA) shown below

R⁹R¹⁰N—[CR^{a}R^{b}]_{b}— (IVA)

where R⁹, R¹⁰, R^{a}, R^{b} and b are as defined above.

Examples of compounds of formula (I) include compounds of formula (IA)

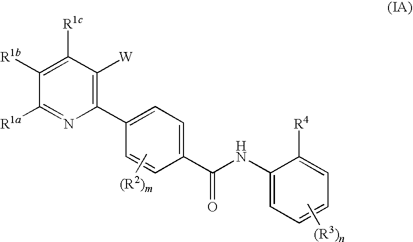

wherein:
R^{1a} is selected from hydrogen, amino, (1-3C)alkyl, N-(1-3C)alkylamino, N,N-di-(1-3C)alkylamino, or a group of the sub-formula II:

R⁵R⁶N—X¹—[CR^{a}R^{b}]_{q}— (II)

wherein:

integer q is 1, 2 or 3;
each R^{a} and R^{b} group present is independently selected from hydrogen, halo, hydroxy or (1-4C)alkyl;

X¹ is selected from a direct bond or —C(O)—; and

R⁵ and R⁶ are each independently selected from hydrogen or (1-3C)alkyl;

and wherein if R^{1a} is a N-(1-3C)alkylamino or N,N-di-(1-3C)alkylamino group, the (1-3C)alkyl moiety is optionally substituted by hydroxy or (1-2C)alkoxy;

R^{1b} is selected from:
(i) hydrogen, (1-6C)alkyl, halo(1-6C)alkyl, hydroxy(1-6C)alkyl, (3-6C)cycloalkyl, (3-6C)cycloalkyl(1-6C) alkyl, (1-6C)alkoxy, (1-6C) alkoxy(1-6C)alkyl, N-(1-6C)alkylsulphamoyl, N,N-di-[(1-6C)alkyl]sulphamoyl; or
(ii) a group of sub-formula III:

R⁷R⁸N—[CR^{a}R^{b}]ₐ—X²— (III)

wherein:

X² is selected from a direct bond, —O— or —C(O)—;

integer a is 0, 1, 2, 3 or 4;

R^{a} and R^{b} are as defined above;
R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IVA

R⁹R¹⁰N—[CR^{a}R^{b}]_{b}— (IVA)

wherein:

integer b is 1, 2 or 3;

R^{a} and R^{b} are as defined above;

R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);

or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by hydroxy, halo, (1-4C)alkyl, carbamoyl, oxo, or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl); or
(iii) a group of the formula V:

R15R¹⁶N—X³—[CR^{a}R^{b}]_{c}— (V)

wherein:

integer c is 0, 1, 2 or 3;

R^{a} and R^{b} are as defined above;

X³ is —C(O)—;

R¹⁵ and R¹⁶ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula VI:

R¹⁷R¹⁸N—[CR^{a}R^{b}]_{d}— (VI)

wherein:

integer d is 1, 2 or 3;

R^{a} and R^{b} are as defined above;

R¹⁷ and R¹⁸ are independently selected from hydrogen, (1-6C)alkyl, (3-6C)cycloalkyl, (3-6C)cycloalkyl(1-6C)alkyl, (1-6C)alkoxy(1-6C)alkyl, or R¹⁷ and R¹⁸ are linked so that, together with the nitrogen atom to which they are attached, they form a 4-, 5-, 6- or 7-membered heterocyclic ring, said heterocyclic ring optionally comprising, in addition to the nitrogen atom to which R¹⁷ and R¹⁸ are attached, one or two further nitrogen atoms, and wherein the heterocyclic ring is optionally substituted by 1, 2 or 3, substituents selected from hydroxy, halo, (1-4C)alkyl, carbamoyl, oxo, or —[CH₂]_{g}—NR¹⁹R²⁰ (wherein g is 0, 1 or 2, and R¹⁹ and R²⁰ are independently selected from hydrogen, (1-6C)alkyl, (3-6C)cycloalkyl or (3-6C)cycloalkyl(1-6C)alkyl);

or R¹⁵ and R¹⁶ are linked so that, together with the nitrogen atom to which they are attached, they form a 4-, 5-, 6- or 7-membered heterocyclic ring, said heterocyclic ring optionally comprising, in addition to the nitrogen atom to which R¹⁵ and R¹⁶ are attached, one or two further nitrogen atoms and the heterocyclic ring is optionally substituted by 1, 2 or 3, substituents selected from hydroxy, halo, (1-4C)alkyl, carbamoyl, oxo, or —[CH₂]ₕ—NR²¹R²² (wherein h is 0, 1 or 2, and R²¹ and R²² are independently selected from hydrogen, (1-6C)alkyl, (3-6C)cycloalkyl or (3-6C)cycloalkyl(1-6C)alkyl);
(iv) a group of the sub-formula VII:

Q-Z—Y— (VII)

wherein:

Y is a direct bond or —[CR^{a}R^{b}]ₓ—, where integer x is 1 to 4 and R^{a} and R^{b} are as defined above;

Z is absent or selected from —O—, —S—, —SO—, —SO₂—, —NH—SO₂—,

—SO₂—NH— or —C(O)—; and

Q is a carbon-linked heterocyclyl or a heterocyclyl-(1-6C)alkyl group, said heterocyclyl or a heterocyclyl-(1-6C)alkyl group being optionally substituted on the heterocyclyl ring by one or more substituent groups (for example 1, 2 or 3), which may be the same or different, selected from halo, oxo, cyano, hydroxy, trifluoromethyl, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, (1-3C)alkoxy(1-3C)alkyl, (1-3C)alkoxycarbonyl, halo(1-3C)alkyl, N-[(1-3C)alkyl]amino, N,N-di-[(1-3C)alkyl]amino, N-[(1-3C)alkoxy(1-3C)alkyl]amino, N,N-di-[(1-3C)alkoxy(1-3C)alkyl]amino, N-[(1-3C)alkoxy(1-3C)alkyl]-N-[(1-3C)alkyl]amino, N-(1-3C)alkylcarbamoyl, N,N-di-[(1-3C)alkyl]carbamoyl, (1-3C)alkylthio, (1-3C)alkylsulphinyl, (1-3C)alkylsulphonyl, N-(1-3C)alkylsulphamoyl, N,N-di-[(1-3C)alkyl]sulphamoyl;

R^{1c} is selected from hydrogen, halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl; integer m is 0, 1, 2, 3 or 4;

R² is halo;

integer n is 0, 1, 2, 3 or 4;

R³ is selected from halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-Di(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;

R⁴ is amino or hydroxy; and

W is fluoro, chloro or bromo;

or a pharmaceutically acceptable salt or pro-drug thereof.

Particular novel compounds of the invention include, for example, benzamide derivatives of the Formula I, or pharmaceutically-acceptable salts or pro-drugs thereof, wherein, unless otherwise stated, each of R^{1a}, R^{1b}, R^{1c}, m, R², n, R³, R⁴ and W has any of the meanings defined hereinbefore or in paragraphs (1) to (22) hereinafter:
(1) R^{1a} is selected from hydrogen, amino, (1-3C)alkyl, N-(1-3C)alkylamino, and N,N-di-(1-3C)alkylamino;

and wherein if R^{1a} is a N-(1-3C)alkylamino or N,N-di-(1-3C)alkylamino group, the (1-3C)alkyl moiety is optionally substituted by hydroxy or (1-2C)alkoxy;
(2) R^{1a} is selected from hydrogen, amino, (1-2C)alkyl, N-(1-2C)alkylamino, and N,N-di-(1-2C)alkylamino;
(3) R^{1a} is hydrogen;
(4) R^{1b} is selected from:

(i) hydrogen, (1-4C)alkyl, halo(1-4C)alkyl, hydroxy(1-4C)alkyl, (3-4C)cycloalkyl, (3-4C)cycloalkyl(1-2C)alkyl, (1-4C)alkoxy, (1-2C)alkoxy(1-2C)alkyl, N-(1-4C)alkylsulphamoyl, N,N-di-[(1-4C)alkyl]sulphamoyl;

(ii) a group of sub-formula III:

R⁷R⁸N—[CR^{a}R^{b}]ₐ—X²— (III)

wherein:
X² is selected from a direct bond, —O— or —C(O)—;
integer a is 0, 1, 2 or 3;
each R^{a} and R^{b} group present is independently selected from hydrogen or (1-2C)alkyl;
   R⁷ and R⁸ are independently selected from hydrogen, (1-4C)alkyl, (3-4C)cycloalkyl, (3-4C)cycloalkyl(1-2C)alkyl, (1-2C)alkoxy(1-2C)alkyl, or a group of sub-formula IVA:

R⁹R¹⁰N—[CR^{a}R^{b}]_{b}— (IVA)
wherein:
   integer b is 1, 2 or 3;
   R^{a} and R^{b} are as defined above;
   R⁹ and R¹⁰ are independently selected from hydrogen, (1-4C)alkyl, (3-4C)cycloalkyl, (3-4C)cycloalkyl(1-2C)alkyl, (1-2C)alkoxy(1-2C)alkyl, or R⁹ and R¹⁰ are linked so that, together with the nitrogen atom to which they are attached, they form a 4-, 5-, or 6-membered heterocyclic ring, wherein said heterocyclic ring optionally comprises, in addition to the nitrogen atom to which R⁹ and R¹⁰ are attached, an additional nitrogen atom, and the heterocyclic ring is optionally substituted by hydroxy, (1-2C)alkyl, oxo, or —[CH₂]ₑ—NR¹¹R¹² (wherein e is 0 or 1, and R¹¹ and R¹² are independently selected from hydrogen, or (1-2C)alkyl);
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by hydroxy, (1-3C)alkyl, oxo, or —[CH₂]_{f}—NR¹³R¹⁴ (wherein f is 0 or 1, and R¹³ and R¹⁴ are independently selected from hydrogen, or (1-2C)alkyl);

(iii) a group of the formula V:

R¹⁵R¹⁶N—X³—[CR^{a}R^{b}]_{c}— (V)

wherein:
integer c is 0, 1 or 2;
R^{a} and R^{b} are as defined above;
X³ is —C(O)—;
R¹⁵ and R¹⁶ are independently selected from hydrogen, (1-4C)alkyl, (3-4C)cycloalkyl, (3-4C)cycloalkyl(1-2C)alkyl, (1-2C)alkoxy(1-2C)alkyl, or a group of formula VI:

R¹⁷R¹⁸N—[CR^{a}R^{b}]_{d}— (VI)
wherein
integer d is 1, 2 or 3;
R^{a} and R^{b} are as defined above;
R¹⁷ and R¹⁸ are independently selected from hydrogen, (1-4C)alkyl, (3-4C)cycloalkyl, (3-4C)cycloalkyl(1-2C)alkyl, (1-2C)alkoxy(1-2C)alkyl, or R¹⁷ and R¹⁸ are linked so that, together with the nitrogen atom to which they are attached, they form a 4-, 5-, or 6-membered heterocyclic ring, wherein said heterocyclic ring optionally comprises, in addition to the nitrogen atom to which R¹⁷ and R¹⁸ are attached, an additional nitrogen atom, and the heterocyclic ring is optionally substituted by hydroxy, (1-2C)alkyl, oxo, or —[CH₂]_{g}—NR¹⁹R²⁰ (wherein g is 0 or 1, and R¹⁹ and R²⁰ are independently selected from hydrogen, or (1-2C)alkyl);
or R¹⁵ and R¹⁶ are linked so that, together with the nitrogen atom to which they are attached, they form a 4-, 5-, or 6-membered heterocyclic ring, wherein said heterocyclic ring optionally comprises, in addition to the nitrogen atom to which R¹⁵ and R¹⁶ are attached, an additional nitrogen atom and the heterocyclic ring is optionally substituted by hydroxy, (1-2C)alkyl, oxo, or —[CH₂]ₕ—NR²¹R²² (wherein h is 0 or 1, and R²¹ and R²² are independently selected from hydrogen, or (1-2C)alkyl);

(iv) a group of the sub-formula VII:

Q-Z—Y— (VII)

wherein:
Y is a direct bond or —[CR^{a}R^{b}]ₓ—, where integer x is 1 to 2 and R^{a} and R^{b} are as defined above;
Z is a direct bond or selected from —O— or —C(O)—; and
Q is a carbon-linked heterocyclyl or a heterocyclyl-(1-2C)alkyl group, said heterocyclyl or a heterocyclyl-(1-2C)alkyl group being optionally substituted on the heterocyclyl ring by one or more substituent groups (for example 1, 2 or 3), which may be the same or different, selected from halo, oxo, amino, (1-2 C)alkyl, (1-2C)alkoxy, (1-2C) alkoxy(1-2C)alkyl, (1-2C)alkoxycarbonyl, halo(1-2C)alkyl, N-[(1-2C)alkyl]amino, and N,N-di-[(1-2C)alkyl]amino;
(5) R^{1b} is selected from:

(i) hydrogen, (1-2C)alkyl, or hydroxy(1-2C)alkyl;

(ii) a group of sub-formula III:

R⁷R⁸N—[CR^{a}R^{b}]ₐ—X²— (III)

wherein:

X² is selected from a direct bond or —O—;

integer a is 1 or 2;

each R^{a} and R^{b} group present is hydrogen;

R⁷ and R⁸ are independently selected from hydrogen, (1-4C)alkyl, (3-4C)cycloalkyl, (3-4C)cycloalkyl(1-2C)alkyl, or a group of sub-formula IVA

R⁹R¹⁰N—[CR^{a}R^{b}]_{b}— (IVA)

wherein:
integer b is 2 or 3;
R^{a} and R^{b} are as defined above;
R⁹ and R¹⁰ are independently selected from hydrogen, (1-2C)alkyl, or R⁹ and
R¹⁰ are linked so that, together with the nitrogen atom to which they are attached, they form a 4-, 5-, or 6-membered heterocyclic ring, wherein the nitrogen atom to which R⁹ and 10 are attached is the only heteroatom present in the ring;
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-3C)alkyl or
—[CH₂]_{f}—NR¹³R¹⁴ (wherein f is 0, and R¹³ and R¹⁴ are independently selected from hydrogen, or (1-2C)alkyl);

(iii) a group of the formula V:

R¹⁵R¹⁶N—X³—[CR^{a}R^{b}]_{c}— (V)
R^{a} and R^{b} are as defined above;
X³ is —C(O)—;
R¹⁵ and R¹⁶ are independently selected from hydrogen, (1-2C)alkyl, or a group of formula VI:

R¹⁷R¹⁸N—[CR^{a}R^{b}]_{d}— (VI)
wherein
   integer d is 2 or 3;
   R^{a} and R^{b} are as defined above;
   R¹⁷ and R¹⁸ are independently selected from hydrogen, (1-2C)alkyl, or R¹⁷ and R¹⁸ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;

(iv) a group of the sub-formula VII:

Q-Z—Y— (VII)

wherein:

Y is a direct bond;

Z is —O—; and

Q is a heterocyclyl-(1-2C)alkyl group optionally substituted on the heterocyclyl ring by one or more substituent groups (for example 1, 2 or 3), which may be the same or different, selected from halo, oxo, amino, (1-2C)alkyl, (1-2C)alkoxy, N-[(1-2C)alkyl]amino, and N,N-di-[(1-2C)alkyl]amino;
(6) R^{1b} is selected from:

(i) hydrogen, (1-6C)alkyl, halo(1-6C)alkyl, hydroxy(1-6C)alkyl,

(ii) a group of sub-formula III:

R⁷R⁸N—[CR^{a}R^{b}]ₐ—X²— (III)
wherein:
X² is a direct bond or —O—;
integer a is 1 or 2;
R^{a} and R^{b} are both hydrogen;
R⁷ and R⁸ are independently selected from hydrogen, (1-4C)alkyl, (3-4C)cycloalkyl, (3-4C)cycloalkyl(1-2C)alkyl, or a group of formula IVA:

R⁹R¹⁰N—[CR^{a}R^{b}]_{b}— (IVA)
wherein:
   integer b is 2;
   R^{a} and R^{b} are as defined above;
   R⁹ and R¹⁰ are independently selected from hydrogen, (1-2C)alkyl, or R⁹ and R¹⁰ are linked so that, together with the nitrogen atom to which they are attached, they form a 4-, 5- or 6-membered heterocyclic ring, and wherein the nitrogen atom to which R⁹ and R¹⁰ are attached is the only heteroatom present in the ring;
or R⁷ and R⁸ are linked so that, together with the nitrogen atom to which they are attached, they form a 4-, 5- or 6-membered heterocyclic ring, which is optionally substituted by (1-3C)alkyl or —[CH₂]_{f}—NR¹³R¹⁴ (wherein f is 0, and R¹³ and R¹⁴ are independently selected from hydrogen, or (1-2C)alkyl), and wherein the nitrogen atom to which R⁹ and R¹⁰ are attached is the only heteroatom present in the ring;

(iii) a group of the formula V:

R¹⁵R¹⁶N—X³—[CR^{a}R^{b}]_{c}— (V)

wherein:
integer c is 0;
X³ is —C(O)—;
R¹⁵ and R¹⁶ are independently selected from hydrogen, (1-2C)alkyl, or a group of formula VI:

R¹⁷R¹⁸N—[CR^{a}R^{b}]_{d}— (VI)
wherein
integer d is 2;
R^{a} and R^{b} are as defined above;
R¹⁷ and R¹⁸ are independently selected from hydrogen, (1-2C)alkyl, or R¹⁷ and R¹⁸ are linked so that, together with the nitrogen atom to which they are attached, they form a 4-, 5- or 6-membered heterocyclic ring, wherein the nitrogen atom to which R¹⁷ and R¹⁸ are attached is the only heteroatom present in the ring;

(iv) a group of the sub-formula VII:

Q-Z—Y— (VII)
wherein:
Y is a direct bond;
Z is —O—; and
Q is heterocyclyl-(1-2C)alkyl, wherein the heterocyclyl group is optionally substituted by (1-2C)alkyl;
(7) R^{1b} is selected from:

(i) hydrogen, (1-2C)alkyl or hydroxy(1-2C)alkyl;

(ii) a group of sub-formula III:

R⁷R⁸N—[CR^{a}R^{b}]ₐ—X²— (III)

wherein:
X² is selected from a direct bond or —O—;
integer a is 1 or 2;
R^{a} and R^{b} are both hydrogen;
R⁷ and R⁸ are independently selected from hydrogen, (1-4C)alkyl, (3-4C)cycloalkyl, (3-4C)cycloalkyl(1-2C)alkyl, or a group of sub-formula IVA:

R⁹R¹⁰N—[CR^{a}R^{b}]_{b}— (IVA)
wherein:
integer b is 2;
R^{a} and R^{b} are as defined above;
R⁹ and R¹⁰ are independently selected from hydrogen, (1-2C)alkyl, or R⁹ and R¹⁹ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;
or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-3C)alkyl or —[CH₂]_{f}—NR¹³R¹⁴ (wherein f is 0, and R¹³ and R¹⁴ are independently selected from hydrogen, or (1-2C)alkyl);

(iii) a group of the formula V:

R¹⁵R¹⁶N—X³—[CR^{a}R^{b}]_{c}— (V)

wherein:
integer c is 0;
R^{a} and R^{b} are as defined above;
X³ is —C(O)—;
R¹⁵ and R¹⁶ are independently selected from hydrogen, (1-2C)alkyl, or a group of formula VI:

R¹⁷R¹⁸N—[CR^{a}R^{b}]_{d}— (VI)

wherein
integer d is 2;
R^{a} and R^{b} are as defined above;
R¹⁷ and R¹⁸ are independently selected from hydrogen, (1-2C)alkyl, or R¹⁷ and R¹⁸ are linked so that, together with the nitrogen atom to which they are attached, they form a 4-, 5-, or 6-membered heterocyclic ring, wherein said heterocyclic ring comprises the nitrogen atom to which R¹⁷ and R¹⁸ are attached is the only heteroatom present;

(iv) a group of the sub-formula VII:

Q-Z—Y— (VII)

wherein:
Y is a direct bond;
Z is —O—; and
Q is a heterocyclyl-methyl group optionally substituted on the heterocyclyl ring by (1-2C)alkyl;
(8) R^{1b} is selected from:

(i) hydrogen, methyl or hydroxymethyl;

(ii) a group of sub-formula III:

R⁷R⁸N—[CR^{a}R^{b}]ₐ—X²— (III)

wherein:
X² is selected from a direct bond, or —O—;
integer a is 1 or 2;
R^{a} and R^{b} are both hydrogen;
R⁷ and R⁸ are independently selected from hydrogen, methyl, ethyl, propyl, isopropyl, butyl, cyclopropyl, cyclobutyl, cyclopropylmethyl, or a group of sub-formula IVA:

R⁹R¹⁰N—[CR^{a}R^{b}]_{b}— (IVA)

wherein:
integer b is 2;
R^{a} and R^{b} are as defined above;
R⁹ and R¹⁰ are independently selected from hydrogen, methyl, ethyl, or R⁹ and R¹⁰ are linked so that, together with the nitrogen atom to which they are attached, they form an azetidinyl, pyrrolidinyl, or piperidinyl ring; or R⁷ and R⁸ are linked so that, together with the nitrogen atom to which they are attached, they form a heterocyclic ring selected from azetidinyl, pyrrolidinyl, piperidinyl, and piperazinyl, and wherein said heterocyclic ring is optionally substituted by methyl, ethyl, propyl, or isopropyl or dimethylamino;

(iii) a group of the formula V:

R¹⁵R¹⁶N—X³—[CR^{a}R^{b}]_{c}— (V)

wherein:
integer c is 0;
X³ is —C(O)—;
R¹⁵ and R¹⁶ are independently selected from hydrogen, methyl, ethyl or a group of formula VI:

R¹⁷R¹⁸N—[CR^{a}R^{b}]_{d}— (VI)
wherein
integer d is 2;
R^{a} and R^{b} are as defined above;
R¹⁷ and R¹⁸ are independently selected from hydrogen, methyl, ethyl or R¹⁷ and R¹⁸ are linked so that, together with the nitrogen atom to which they are attached, they form a pyrrolidinyl ring;

(iv) a group of the sub-formula VII:

Q-Z—Y— (VII)
wherein:
Y is a direct bond;
Z is —O—; and
Q is a 1-methylpyrrolidin-2-yl ring;
(9) R^{1b} is selected from hydrogen, hydroxymethyl, (methylamino)methyl, (ethylamino)methyl, (propylamino)methyl, (isopropylamino)methyl, (cyclopropylamino)methyl, (butylamino)methyl, (cyclobutylamino)methyl, (cyclopentylamino)methyl, (1-methylpropylamino)methyl, (2-methylpropylamino)methyl, 1-(methylamino)ethyl, 2-(methylamino)ethyl, 2-(ethylamino)ethyl, 3-(methylamino)propyl, (di-methylamino)methyl, (di-ethylamino)methyl, Rethyl)(methyl)amino]methyl, [(isopropyl)(methyl)amino]methyl, [(propyl)(methyl)amino[methyl, [(cyclopropylmethyl)amino]methyl, [(cyclobutylmethyl)(methyl)amino]methyl, [(2-methoxyethyl)(methyl)amino]methyl, [(isopropyl)(2-methoxyethyl)amino]methyl, [(2-methoxyethyl)amino]methyl, [(ethyl)(2-methoxyethyl)amino]methyl, [(2-hydroxyethyl)(ethyl)amino]methyl, [(2-hydroxyethyl)(methyl)amino]methyl, {[2-(di-methylamino)ethyl]amino}methyl, {[2-(di-ethylamino)ethyl]amino}methyl, {[2-(di-methylamino)ethyl][methyl]amino}methyl, {[2-(di-ethylamino)ethyl][methyl]amino}methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4-ethylpiperazin-1-yl)methyl, (4-isopropylpiperazin-1-yl)methyl, 2-(dimethylamino)ethoxy, 2-pyrrolidin-1-ylethoxy, and 2-(4-methylpiperazin-1-yl)ethoxy, N-[2-(diethylamino)ethyl]carbamoyl, N-(2-pyrrolidin-1-ylethyl)carbamoyl, and N[2-(dimethylamino)ethyl)]-N-(methyl)carbamoyl, or (1-methylpyrrolidin-2-yl)methoxy;
(10) R^{1b} is selected from hydrogen, hydroxymethyl, (methylamino)methyl, (ethylamino)methyl, (propylamino)methyl, (isopropylamino)methyl, (butylamino)methyl, (cyclobutylamino)methyl, (2-methylpropylamino)methyl, [(cyclopropylmethyl)amino]methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4-ethylpiperazin-1-yl)methyl, (4-isopropylpiperazin-1-yl)methyl, 2-(dimethylamino)ethoxy, 2-pyrrolidin-1-ylethoxy, and 2-(4-methylpiperazin-1-yl)ethoxy, N-[2-(diethylamino)ethyl]carbamoyl, N-(2-pyrrolidin-1-ylethyl)carbamoyl, and N-[2-(dimethylamino)ethyl)]-N-(methyl)carbamoyl, and (1-methylpyrrolidin-2-yl)methoxy;
(11) R^{1b} is selected from hydroxymethyl, (methylamino)methyl, (ethylamino)methyl, (propylamino)methyl, (isopropylamino)methyl, (butylamino)methyl, cyclobutylamino)methyl, (2-methylpropylamino)methyl, [(cyclopropylmethyl)amino]methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-ethylpiperazin-1-yl)methyl, (4-isopropylpiperazin-1-yl)methyl, 2-(dimethylamino)ethoxy, 2-pyrrolidin-1-ylethoxy, and 2-(4-methylpiperazin-1-yl)ethoxy, N-[2-(diethylamino)ethyl]carbamoyl, N-(2-pyrrolidin-1-ylethyl)carbamoyl, and N-[2-(dimethylamino)ethyl)]-N-(methyl)carbamoyl, or (1-methylpyrrolidin-2-yl)methoxy;
(12) R^{1b} is a group of sub-formula III:

R⁷R⁸N—[CR^{a}R^{b}]ₐ—X²— (III)

wherein:

X² is selected from a direct bond, —O— or —C(O)—;

integer a is 0, 1, 2, 3 or 4;

R^{a} and R^{b} are both hydrogen;

R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IVA:

R⁹R¹⁰N—[CR^{a}R^{b}]_{b}— (IVA)

wherein:

integer b is 1, 2 or 3;

R^{a} and R^{b} are as defined above;

R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two heteroatoms selected from N, O or S, and wherein the heterocyclic ring is optionally substituted by hydroxy, halo, (1-4C)alkyl, carbamoyl, oxo, or

—[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);

or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two heteroatoms selected from N, O or S, and wherein the heterocyclic ring is optionally substituted by hydroxy, halo, (1-4C)alkyl, carbamoyl, oxo, or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);
(13) R^{1b} is a group of sub-formula III:

R⁷R⁸N—[CR^{a}R^{b}]ₐ—X²— (III)

wherein:

X² is selected from a direct bond or —O—;

integer a is 1, 2 or 3;

R^{a} and R^{b} are both hydrogen;

R⁷ and R⁸ are independently selected from hydrogen, (1-4C)alkyl, (3-4C)cycloalkyl, (3-4C)cycloalkyl(1-2C)alkyl, or a group of sub-formula IVA:

R⁹R¹⁰N—[CR^{a}R^{b}]_{b}— (IVA)

wherein:

integer b is 2 or 3;

R^{a} and R^{b} are as defined above;

R⁹ and R¹⁰ are independently selected from hydrogen, (1-2C)alkyl, or R⁹ and R¹⁰ are linked so that, together with the nitrogen atom to which they are attached, they form a 4-, 5-, or 6-membered heterocyclic ring, wherein the nitrogen atom to which R⁹ and R¹⁰ are attached is the only heteroatom present in the ring;

or R⁷ and R⁸ are linked so that, together with the nitrogen atom to which they are attached, they form a 4-, 5-, or 6-membered heterocyclic ring, wherein said heterocyclic ring optionally comprises, in addition to the nitrogen atom to which R⁷ and R⁸ are attached, an additional nitrogen atom, and the heterocyclic ring is optionally substituted by (1-3C)alkyl or —[CH₂]_{f}—NR¹³R¹⁴ (wherein f is 0, and R¹³ and R¹⁴ are independently selected from hydrogen, or (1-2C)alkyl);
(14) R^{1b} is selected from (methylamino)methyl, (ethylamino)methyl, (propylamino)methyl, (isopropylamino)methyl, (butylamino)methyl, (cyclobutylamino)methyl, (2-methylpropylamino)methyl, [(cyclopropylmethyl)amino]methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4-ethylpiperazin-1-yl)methyl, (4-isopropylpiperazin-1-yl)methyl, 2-(dimethylamino)ethoxy, 2-pyrrolidin-1-ylethoxy, or 2-(4-methylpiperazin-1-yl)ethoxy;
(15) R^{1c} is selected from hydrogen, amino, (1-3C)alkyl, N-(1-3C)alkylamino, and N,N-di-[(1-3C)alkyl]amino;
(16) R^{1c} is hydrogen;
(17) m is 0;
(18) n is 0;
(19) R⁴ is amino;
(20) W is fluoro or chloro;
(21) W is fluoro;
(22) W is chloro;
(23) W is bromo;
(24) R^{1b} is selected from hydrogen, hydroxymethyl, or a group of sub-formula III:

R⁷R⁸N—[CR^{a}R^{b}]ₐ—X²— (III)

wherein:

X² is selected from a direct bond, —O— or —C(O)—;

a is 0, 1, 2, 3 or 4;

each R^{a} and R^{b} group is independently selected from hydrogen, methyl or ethyl;

R⁷ and R⁸ are independently selected from hydrogen, (1-6C)alkyl, (3-6C)cycloalkyl, (3-6C)cycloalkyl(1-6C)alkyl, (1-6C)alkoxy(1-6C)allcyl, or a group of formula IV:

R⁹R¹⁰N—[CR^{a}R^{b}]_{b}—X⁴— (IV)

wherein:

b is 1, 2 or 3;

R^{a} and R^{b} are as defined above;

X⁴ is a direct bond or —C(O)—;

R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);

or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by hydroxy, halo, (1-4C)alkyl, carbamoyl, oxo, (2-4C)alkenyl, (2-4C)alkynyl, (1-4C)alkoxy, (1-4C)alkoxy(1-4C) alkyl, (1-4C)alkyl-S(O)_{q}— (where q is 0, 1 or 2), a 5- or 6-membered heterocyclic ring comprising one to three heteroatoms selected from N, O or S, or a group —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);
(25) R^{1b} is selected from hydrogen, hydroxymethyl, or a group of sub-formula III:

R⁷R⁸N—[CR^{a}R^{b}]ₐ—X²— (III)

wherein:

X² is selected from a direct bond, —O— or —C(O)—;

a is 0, 1, 2, 3 or 4;

each R^{a} and R^{b} group is independently selected from hydrogen or methyl; R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IV:

R⁹R¹⁰N—[CR^{a}R^{b}]_{b}—X⁴— (IV)

wherein:

b is 1, 2 or 3;

R^{a} and R^{b} are as defined above;

X⁴ is a direct bond or —C(O)—;

R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further nitrogen atoms, and wherein said heterocyclic ring is optionally substituted by hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-4C)alkyl, (3-6C)cycloalkyl or (3-6C)cycloalkyl(1-2C)alkyl);

or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further nitrogen atoms, and wherein said heterocyclic ring is optionally substituted by hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), or a group —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);
(26) R^{1b} is selected from hydrogen, hydroxymethyl, or a group of sub-formula III:

R⁷R⁸N—[CR^{a}R^{b}]ₐ—X²— (III)

wherein:

X² is selected from a direct bond, —O— or —C(O)—;

a is 0, or 1;

each R^{a} and R^{b} group is hydrogen;

R⁷ and R⁸ are independently selected from hydrogen, (1-6C)alkyl, (3-6C)cycloalkyl, (3-6C)cycloalkyl(1-2C)alkyl, (1-4C)alkoxy(1-4C)alkyl, or a group of formula IV:

R⁹R¹⁰N—[CR^{a}R^{b}]_{b}—X⁴— (IV)

wherein:

b is 1, 2 or 3;

R^{a} and R^{b} are as defined above;

X⁴ is a direct bond;

R⁹ and R¹⁰ are independently selected from hydrogen, (1-4C)alkyl, (3-6C)cycloalkyl, (3-6C)cycloalkyl(1-2C)alkyl, (1-4C)alkoxy(1-4C)alkyl, or R⁹ and R¹⁰ are linked so that, together with the nitrogen atom to which they are attached, they form a 4-, 5-, or 6-membered heterocyclic ring, and wherein said heterocyclic ring is optionally substituted by hydroxy, halo, or (1-2C)alkyl,

or R⁷ and R⁸ are linked so that, together with the nitrogen atom to which they are attached, they form a 4-, 5-, 6-membered heterocyclic ring, said heterocyclic ring optionally comprising, in addition to the nitrogen atom to which R⁷ and R⁸ are attached, one further nitrogen atom, and wherein said heterocyclic ring is optionally substituted by hydroxy, halo, (1-4C)alkyl, carbamoyl, oxo, (1-4C)alkoxy, (1-4C)alkoxy(1-4C)alkyl, (1-4C)alkyl-S(O)_{q}— (where q is 0, 1 or 2), or a group —[CH₂]_{f}—NR¹³R¹⁴ (wherein f is 0, 1 or 2, and R¹³ and R¹⁴ are independently selected from hydrogen, or (1-4C)alkyl);
(27) R^{1b} is hydrogen or a group of formula (III) having anyone definitions set out herein;
(28) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S, or a group —[CH₂]_{f}—NR¹³R¹⁴ or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl);
(29) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S;
(30) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by (1-4C)alkoxy(1-4C)alkyl or (1-4C)alkyl-S(O)_{q}— (where q is 0, 1 or 2);
(31) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by (1-4C)alkoxy(1-4C)alkyl;
(32) R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by (1-4C)alkyl-S(O)_{q}— (where q is 0, 1 or 2), and preferably is 2;
(33) R^{1b} is a group of sub-formula (III) where a least one group R⁷ or R⁸ is a group of sub-formula (IV) where X³ is a direct bond;
(34) R^{1b} is a group of sub-formula (III) where a least one group R⁷ or R⁸ is a group of sub-formula (IV) where X³ is a —(CO)—;
(35) R^{1b} is a group of sub-formula (III) where a least one group R⁷ or R⁸ is a group of sub-formula (IV) where b is 2;
(36) R^{1b} s a group of sub-formula (III) where at least one group R⁷ or R⁸ is a (1-6C)alkoxy(1-6C)alkyl;
(37) R^{1b} is a group of sub-formula (III) where at least one group R⁷ or R⁸ is (1-4C)alkoxy(1-4C)alkyl, wherein the alkyl or alkoxy moiety is optionally branched;
(38) R^{1b} a group of sub-formula (III) where at least one group R⁷ or R⁸ is (1-4C)alkoxy(1-4C)alkyl selected from methoxyethyl, methoxypropyl, ethoxyethyl, ethoxypropyl, propoxyethyl, or 2-methoxy-2,2 dimethylethyl.
(39) R^{1b} is selected from hydrogen, hydroxymethyl, (methylamino)methyl, (ethylamino)methyl, (propylamino)methyl, (isopropylamino)methyl, (cyclopropylamino)methyl, (butylamino)methyl, (cyclobutylamino)methyl, (cyclopentylamino)methyl, (1-methylpropylamino)methyl, (2-methylpropylamino)methyl, 1-(methylamino)ethyl, 2-(methylamino)ethyl, 2-(ethylamino)ethyl, 3-(methylamino)propyl, (di-methylamino)methyl, (di-ethylamino)methyl, [(ethyl)(methyl)amino]methyl, [(isopropyl)(methyl)amino]methyl, [(propyl)(methyl)amino]methyl, [(cyclopropylmethyl)amino]methyl, [(cyclobutylmethyl)(methyl)amino]methyl, [(2-methoxyethyl)(methyl)amino]methyl, [(isopropyl)(2-methoxyethyl)amino]methyl, [(2-methoxyethyl)amino]methyl, [(ethyl)(2-methoxyethyl)amino]methyl, {[2-(di-methylamino)ethyl]amino}methyl, [2-(di-ethylamino)ethyl]amino}methyl, {[2-(di-methylamino)ethyl][methyl]amino}methyl, {[2-(di-ethylamino)ethyl][methyl]amino}methyl, [(2-pyrrolidin-1-ylethyl)amino]methyl, azetidin-1-ylmethyl, pyrrolidin-1-ylmethyl, [3-(dimethylamino)pyrrolidin-1-yl]methyl, (4-methylpiperazin-1-yl)methyl, (4- ethylpiperazin-1-yl)methyl, (4-isopropylpiperazin-1-yl)methyl, 2-(dimethylamino)ethoxy, 2-pyrrolidin-1-ylethoxy, and 2-(4-methylpiperazin-1-yl)ethoxy, N-2-[dimethylamino]ethyl-N-methyl-carbamoyl, N-2-[pyrrolidin-1-yl]ethyl-carbamoyl, N-2-[diethylamino] ethyl-carbamoyl, (3-methoxypropyl)aminolmethyl, [(3-ethoxypropyl)amino]methyl, [(2-ethoxyethyl)amino]methyl, [3-(methylsulfonyl)pyrrolidin-1-yl]methyl, [4-(2-methoxyethyl)piperazin-1-yl]methyl, (1-methylpyrrolidin-2-yl)methoxy and [(2-propoxyethyl)amino]methyl.
(40) R^{1b} is selected from hydrogen, N-2-[dimethylamino]ethyl-N-methyl-carbamoyl, N-2-[pyrrolidin-1-yl]ethyl-carbamoyl, (methylamino)methyl, (ethylamino)methyl, (propylamino)methyl, (isopropylamino)methyl, (cyclopropylmethyl)amino]methyl, [diethylamino]ethyl-carbamoyl), hydroxymethyl, (4-methylpiperazin-1-yl)methyl], (4-ethylpiperazin-1-yl)methyl, (4-isopropylpiperazin-1-yl)methyl, pyrrolidin-1-ylmethyl, [3-(dimethylamino)pyrrolidin-1-yl]methyl, (azetidin-1-ylmethyl), (butylamino)methyl, (isobutylamino)methyl, (cyclobutylamino)methyl, (2-pyrrolidin-1-ylethyl)amino]methyl, 2-(dimethylamino)ethoxy, (4-methylpiperazin-1-yl)ethoxy, 2-pyrrolidin-1-ylethoxy, [(2S)-1-methylpyrrolidin-2-yl]methoxy, (3-methoxypropyl)amino]methyl, (2-methoxyethyl)amino]methyl, (3-ethoxypropyl)amino]methyl, (2-ethoxyethyl)amino, 3-(methylsulfonyl)pyrrolidin-1-yl]methyl, 2-methoxyethyl and (2-propoxyethyl)amino]methyl.
(41) When R^{1b} is a group of sub-formula (III) and one of R⁷ or R⁸ is a group of sub-formula (IV), then R⁹ and R¹⁰ together with the nitrogen atom to which they are attached form a 4-, 5-, 6- or 7-membered heterocyclic ring, said heterocyclic ring optionally comprising, in addition to the nitrogen atom to which R⁹ and R¹⁰ are attached, one or two further nitrogen atoms, wherein the ring may be optionally substituted as defined above.

Suitably, R^{1a} is hydrogen, amino or (1-3C)alkyl, particularly hydrogen.

Suitably, R^{1c} is hydrogen, amino or (1-3C)alkyl, particularly hydrogen.

Suitably, integer m is 0, 1, 2 or 3, particularly 0.

Suitably, R², when present, is fluoro or chloro, particularly fluoro.

Suitably, integer n is 0, 1, 2 or 3, particularly 0.

Suitably, R³, when present, is hydroxy, fluoro or chloro, particularly fluoro.

Suitably, R⁴ is amino.

Suitably, W is fluoro or chloro, especially chloro.

Suitably, R^{1b} is a group of sub-formula III as hereinbefore defined (and particularly as defined in any one of paragraphs (4) to (14) above).

When R^{1b} is a group of sub-formula (III), and at least one of R⁷ or R⁸ is a group of sub-formula (IV), integer b is suitably 1, 2 or 3, particularly 2 or 3 and most particularly 2.

Preferably when R⁷ and R⁸ are not linked so as to form a ring, one of R⁷ or R⁸ is hydrogen or (1-6C)alkyl, such as methyl, and most preferably one of R⁷ or R⁸ is hydrogen.

In particular, one of R⁷ or R⁸ is hydrogen or (1-6C)alkyl, such as methyl, but in particular is hydrogen, and the other is (1-6C)alkyl, (3-6C)cycloalkyl, (3-6C)cycloalkyl(1-6C)alkyl, (1-6C)alkoxy(1-6C)alkyl, (2-3C)alkenyl, (2-3C)alkynyl, or a group of formula (IV) as defined above. Most suitably one of R⁷ or R⁸ is hydrogen or (1-6C)alkyl, such as methyl, but in particular is hydrogen, and the other is (1-6C)alkyl, such as methyl, ethyl, propyl or iso-propyl, or (1-6C)alkoxy(1-6C)alkyl such as rnethoxyethyl, ethoxyethyl, methoxypropyl.

In a particular embodiment, R^{1b} is a group of sub-formula (III) where R⁷ and R⁸ are linked so that, together with the nitrogen atom to which they are attached, they form a 4-, 5-, 6- or 7-membered heterocyclic ring, said heterocyclic ring may optionally contain one or two further heteroatoms selected from N, O or S, and be optionally substituted as described above. In particular, R⁷ and R⁸ are linked so that, together with the nitrogen atom to which they are attached, they form a 4-, 5- or 6-membered hetercyclic ring which optionally contains one or two further N atoms. Particular examples for rings formed by R⁷ and R⁸ include azetidinyl, pyrrolidinyl, piperidinyl or piperazinyl, and in particular azetidinyl, pyrrolidinyl, or piperazinyl.

Suitably rings R⁷ and R⁸ and the nitrogen atom to which they are attached are substituted by one or more groups, suitably from one to three groups, and most preferably one group which are selected from those defined above. Particular examples of such substituents include (1-4C)alkyl, —[CH₂]_{f}—NR¹³R¹⁴ (wherein f is 0), and R¹³ and R¹⁴ are independently selected from (1-6C)alkyl, (2-4C)alkynyl, (1-4C)alkoxy(1-4C)alkyl, or (1-4C)alkyl-S(O)₂—. Other particular examples of substitutents include 5- or 6-membered heterocyclic rings comprising one to three heteroatoms selected from N, O or S, and in particular, 5- or 6-membered heterocyclic rings containing one or two nitrogen atoms, for example, pyrrolidinyl, piperidinyl, piperazinyl, pyrrolyl, pyrazinyl or pyridyl, and in particular pyrrolidinyl, piperidinyl or piperazinyl.

Suitably, when X² is carbonyl, R⁷ and R⁸ together with the nitrogen atom to which they are attached are other than an optionally substituted 4-, 5-, 6- or 7-membered heterocyclic ring, said heterocyclic ring optionally comprising, in addition to the nitrogen atom to which R⁷ and R⁸ are attached, one or two further heteroatoms selected from N, O or S. In particular, when X² is carbonyl R⁷ and R⁸ together with the nitrogen atom to which they are attached are other than 6-membered heterocyclic ring, said heterocyclic ring comprising, in addition to the nitrogen atom to which R⁷ and R⁸ are attached, one further heteroatoms selected from N and wherein the ring is substituted by (1-4C)alkyl. Most preferably, when X² is carbonyl, R⁷ and R⁸ together with the nitrogen to which they are attached is other than methylpyrazine.

Suitably also, when X² is a group —C(O)—, at least one of R⁷ or R⁸ is a group of formula IV or IVA as defined above.

Suitably also, when X² is carbonyl and one of R⁷ or R⁸ is a group of sub-formula (IV) where X⁴ is a direct bond, then R⁹ and R¹⁰ do not together fowl a morpholine group.

A particular group of compounds according to the present invention have the general formula (IX) shown below

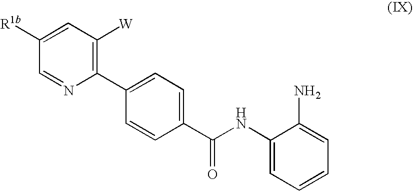

wherein

W is fluoro, chloro or bromo; and

R^{1b} is has any one of the definitions set out hereinbefore (and is particularly as defined in any one of paragraphs (4) to (14) or (24) to (41) above).

In a particular sub-group of compounds of formula (IX), W is fluoro and R^{1b} has any one of the definitions set out hereinbefore (and is particularly as defined in any one of paragraphs (4) to (14) or (24) to (41) above).

In yet another particular sub-group of compounds of formula (IX), W is chloro and R^{1b} has any one of the definitions set out hereinbefore (and is particularly as defined in any one of paragraphs (4) to (14) or (24) to (41) above).

In another particular group of compounds of formula (IX), W is bromo and R^{1b} has any one of the definitions set out hereinbefore (and is particularly as defined in any one of paragraphs (4) to (14) or (24) to (41) above).

In a particular compound of formula (IX), W is bromo and R^{1b} is hydrogen.

A further particular group of compounds of the present invention have the general formula (X)

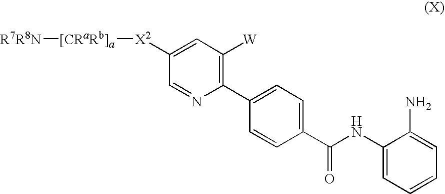

wherein: W is fluoro, chloro or bromo;

and R⁷, R⁸, R^{a}, R^{b}, a and X² each have any one of the definitions set out hereinbefore (and are particularly as defined for the group of sub-formula III in any one of paragraphs (4) to (8), (12), (13), (24) to (26) and (28) to (38) above).

In the compounds of formula (X), W is suitably fluoro or chloro, especially chloro.

A particular sub-group of compounds of formula (X) have the general structural formula (XI):

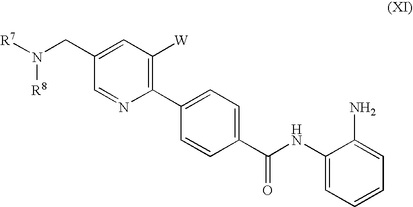

wherein:

W is fluoro, chloro or bromo;

and R⁷ and R⁸ have any one of the definitions set out hereinbefore (and are particularly as defined for the group of sub-formula III in any one of paragraphs (4) to (8), (12), (13), (24) to (26) and (28) to (38) above).

In the compounds of formula (XI), W is suitably fluoro or chloro, especially chloro.

A further particular sub-group of compounds of formula (X) have the general structural formula (XII):

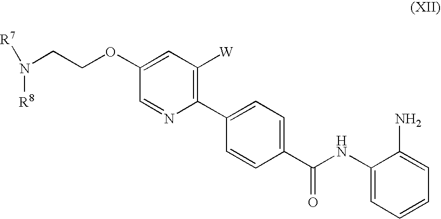

wherein:

W is fluoro, chloro or bromo;

and R⁷ and R⁸ have any one of the definitions set out hereinbefore (and are particularly as defined for the group of sub-formula III in any one of paragraphs (4) to (8), (12), (13), (24) to (26) and (28) to (38) above).

In the compounds of formula (XII), W is suitably fluoro or chloro, especially chloro.

A further particular group of compounds of the invention have the general structural formula (XIII):

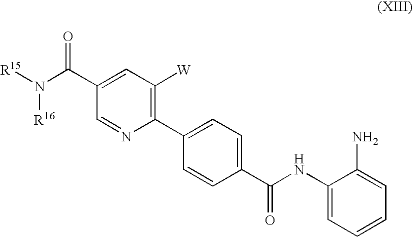

wherein:

W is fluoro, chloro or bromo;

and R¹⁵ and R¹⁶ have any one of the definitions set out hereinbefore (and are particularly as defined for the group of sub-formula VI in any one of paragraphs (4) to (8) above).

In the compounds of formula (XIII), W is suitably fluoro or chloro, especially chloro.

Further novel compounds of the invention include any one of the following:
N-(2-aminophenyl)-4-(3-chloropyridin-2-yl)benzamide;
N-(2-aminophenyl)-4-[3-chloro-5-(N-2-[dimethylamino]ethyl-N-methyl-carbamoyl)-pyridin-2-yl]benzamide (alternative name: 6-(4-{[(2-aminophenyl)amino]carbonyl}phenyl)-5-chloro-N-[2-(dimethylamino)ethyl]-N-methylnicotinamide);
N-(2-aminophenyl)-4-[3-chloro-5-(N-2-[pyrrolidin-1-yl]ethyl-carbamoyl)-pyridin-2-yl]benzamide (alternative name: 6-(4-{[(2-aminophenyl)amino]carbonyl}phenyl)-5-chloro-N-(2-pyrrolidin-1-ylethyl)nicotinamide);
N-(2-aminophenyl)-4-(3-bromopyridin-2-yl)benzamide;
N-(2-aminophenyl)-4-{3-chloro-5-[(methylamino)methyl]pyridin-2-yl}benzamide;
N-(2-aminophenyl)-4-{3-chloro-5-[(ethylamino)methyl]pyridin-2-yl}benzamide;
N-(2-aminophenyl)-4-{3-chloro-5-[(propylamino)methyl]pyridin-2-yl}benzamide;
N-(2-aminophenyl)-4-{3-chloro-5-[(isopropylamino)methyl]pyridin-2-yl}benzamide;
N-(2-aminophenyl)-4-(3-chloro-5-{[(cyclopropylmethyl)amino]methyl}pyridin-2-yl)benzamide;
N-(2-aminophenyl)-4-[3-chloro-5-(N-2-[diethylamino]ethyl-carbamoyl)-pyridin-2-yl]benzamide (alternative name: 6-(4-{[(2-aminophenyl)amino]carbonyl}phenyl)-5-chloro-N-[2-(diethylamino)ethyl]nicotinamide;
N-(2-aminophenyl)-4-[3-chloro-5-(hydroxymethyl)pyridin-2-yl]benzamide;
N-(2-aminophenyl)-4-{3-chloro-5-[(4-methylpiperazin-1-yl)methyl]pyridin-2-yl}benzamide;
N-(2-aminophenyl)-4-{3-chloro-5-[(4-ethylpiperazin-1-yl)methyl]pyridin-2-yl}benzamide;
N-(2-aminophenyl)-4-{3-chloro-5-[(4-isopropylpiperazin-1-yl)methyl]pyridin-2-yl}benzamide;
N-(2-aminophenyl)-4-[3-chloro-5-(pyrrolidin-1-ylmethyl)pyridin-2-yl]benzamide;
N-(2-aminophenyl)-4-(3-chloro-5-{[(3S)-3-(dimethylamino)pyrrolidin-1-yl]methyl}pyridin-2-yl)benzamide;
N-(2-aminophenyl)-4-(3-chloro-5-{[(3R)-3-(dimethylamino)pyrrolidin-1-yl]methyl}pyridin-2-yl)benzamide;
N-(2-aminophenyl)-4-[5-(azetidin-1-ylmethyl)-3-chloropyridin-2-yl]benzamide;
N-(2-aminophenyl)-4-{5-[(butylamino)methyl]-3-chloropyridin-2-yl}benzamide;
N-(2-aminophenyl)-4-{3-chloro-5-[(isobutylamino)methyl]pyridin-2-yl}benzamide;
N-(2-aminophenyl)-4-{3-chloro-5-[(cyclobutylamino)methyl]pyridin-2-yl}benzamide;
N-(2-aminophenyl)-4-(3-chloro-5-{[(2-pyrrolidin-1-ylethyl)amino]methyl}pyridin-2-yl)benzamide;
N-(2-aminophenyl)-4-{3-chloro-5-[2-(dimethylamino)ethoxy]pyridin-2-yl}benzamide;
N-(2-aminophenyl)-4-{3-chloro-5-[2-(4-methylpiperazin-1-yl)ethoxy]pyridin-2-yl}benzamide;
N-(2-aminophenyl)-4-[3-chloro-5-(2-pyrrolidin-1-ylethoxy)pyridin-2-yl]benzamide;
N-(2-aminophenyl)-4-(3-chloro-5-{[(2S)-1-methylpyrrolidin-2-yl]methoxy}pyridin-2-yl)benzamide;
N-(2-aminophenyl)-4-[5-(azetidin-1-ylmethyl)-3-fluoropyridin-2-yl]benzamide;
N-(2-aminophenyl)-4-{3-fluoro-5-[(4-isopropylpiperazin-1-yl)methyl]pyridin-2-yl}benzamide;
N-(2-aminophenyl)-4-{5-[(4-ethylpiperazin-1-yl)methyl]-3-fluoropyridin-2-yl}benzamide;
N-(2-aminophenyl)-4-(3-chloro-5-{[(3-methoxypropyl)amino]methyl}pyridin-2-yl)benzamide;
N-(2-aminophenyl)-4-(3-chloro-5-{[(2-methoxyethyl)amino]methyl}pyridin-2-yl)benzamide;
N-(2-aminophenyl)-4-(3-chloro-5-{[(3-ethoxypropyl)amino]methyl}pyridin-2-yl)benzamide;
N-(2-aminophenyl)-4-(3-chloro-5-{[(2-ethoxyethyl)amino]methyl}pyridin-2-yl)benzamide;
N-(2-aminophenyl)-4-(3-chloro-5-{[3-(methylsulfonyl)pyrrolidin-1-yl]methyl}pyridin-2-yl)benzamide;
N-(2-aminophenyl)-4-(3-chloro-5-{[4-(2-methoxyethyl)piperazin-1-yl]methyl}pyridin-2-yl)benzamide; and
N-(2-aminophenyl)-4-(3-chloro-5-{[(2-propoxyethyl)amino]methyl}pyridin-2-yl)benzamide.

Another aspect of the present invention provides a process for preparing a compound of formula (I) or a pharmaceutically acceptable salt or pro-drug form thereof (wherein R^{1a}, R^{1b}, R^{2c}, R², R³, R⁴, m, n and W are, unless otherwise specified, as hereinbefore defined), said process comprising the steps of:

(a) the reaction of a compound of the formula (A)

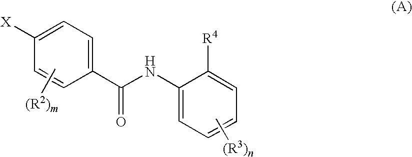

wherein X is a reactive group, with a compound of the formula (B)

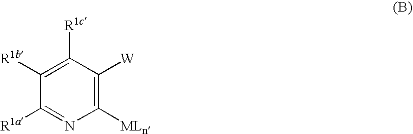

wherein

R^{1a′} K is a group R^{1a} as hereinbefore defined or a precursor thereof,

R^{1b′} is a group R^{1b} as hereinbefore defined or a precursor thereof,

R^{1c′} is a group R^{1c} as hereinbefore defined or a precursor thereof,

M is a metal,

L is a ligand, and

integer n′ is 0 to 3;

and wherein if any one of said groups R^{1c′}, R^{1b′} or R^{1c′} is a precursor for a R^{1a}, R^{1b} or R^{1c} group respectively, then said process thereafter comprises a step of converting the compound formed by the reaction of a compound of the formula (A) with a compound of the formula (B) to a compound of formula (I) (by converting the precursor of any one of groups R^{1a}, R^{1b} or R^{1c} to the appropriate R^{1a}, R^{1b} or R^{1c} group); or

(b) The reaction of a compound of the formula (C)

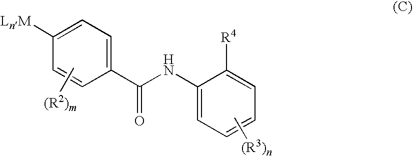

wherein M, L and integer n′ are as defined above, with a compound of the formula (D)

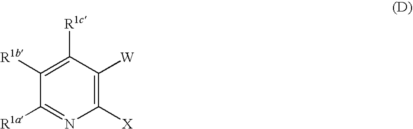

wherein R^{1a′}, R^{1b′} and R^{1c′} are as defined above and X is a reactive group; and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (C) with a compound of the formula (D) to a compound of formula (I) (by converting the precursor of any one of groups R^{1a}, R^{1b} or R^{1c} to the appropriate R^{1a}, R^{1b} or R^{1c} group); or
(c) the reaction, under suitable amide coupling conditions or in the presence of a suitable amide coupling reagent, in particular4-(4,6-dimethoxy-1,3,5-triazinyl-2-yl)-4-methylmorpholinium chloride, of a compound of the formula (E)

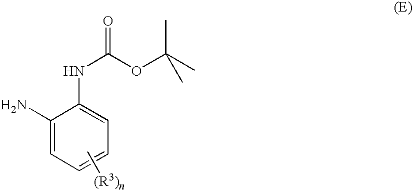

with a compound of the formula (F)

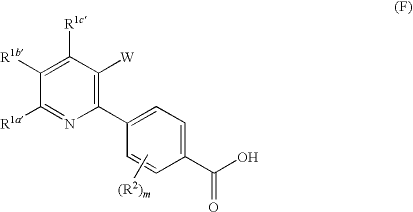

wherein R^{1a′}, R^{1b′} and R^{1c′} are as defined above, and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (E) with a compound of the formula (F) to a compound of formula (I) (by converting the precursor of any one of groups R^{1a}, R^{1b} or R^{1c} to the appropriate R^{1a}, R^{1b} or R^{1c} group);

and thereafter if necessary:

i) converting a compound of the formula (I) into another compound of the formula (I); and/or

ii) removing any protecting groups.

A suitable base for process (a), (b) or (c) is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal hydride, for example sodium hydride, or an alkaline metal hydrogencarbonate such as sodium hydrogencarbonate, or a metal alkoxide such as sodium ethoxide.

A suitable reactive group X is, for example, a halo or a sulphonyloxy group, for example a chloro, bromo, iodo, methanesulphonyloxy, trifluoromethanesulphonyloxy or toluene-4-sulphonyloxy group.

The reactions are conveniently carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran, 1,2-dimethoxyethane or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reactions are conveniently carried out at a temperature in the range, for example, 10 to 250° C., preferably in the range 40 to 80° C.

Metal M may be any metal that is known in the literature to form organometallic compounds that undergo catalytic cross coupling reactions. Examples of suitable metals include boron, tin, zinc, magnesium.

A suitable value for n′ is dependent on the metal M, but is usually in the range 0-3.

Suitable values for the ligand L, when present, include, for example, a hydroxy, a halo, (1-4C)alkoxy or (1-6C)alkyl ligand, for example a hydroxy, bromo, chloro, fluoro, iodo, methoxy, ethoxy, propoxy, isopropoxy, butoxy, methyl, ethyl, propyl, isopropyl or butyl ligand or, where n is 2 and M is boron, the two ligands present may be linked such that, together with the boron atom to which they are attached, they form a ring.

Suitably, the group MLₙ is a group of the formula —BL¹L², where B is boron and L¹ and L² are as defined for ligand L above. In particular, the ligands L¹ and L² may be linked such that, together with the boron atom to which they are attached, they form a ring. For example, L¹ and L² together may define an oxy-(2-4C)alkylene-oxy group, for example an oxyethyleneoxy, a —O—C(CH₃)₂C(CH₃)₂—O— group or an oxypropyleneoxy group such that, together with the boron atom to which they are attached, they form a cyclic boronic acid ester group.

A suitable catalyst for process (a) or (b) includes, for example, a metallic catalyst such as a palladium(0), palladium(II), nickel(0) or nickel(II) catalyst, for example tetrakis(triphenylphosphine)palladium(0), palladium(II) chloride, palladium(II) bromide, bis(triphenylphosphine)palladium(II) chloride, tetrakis(triphenylphosphine)nickel(0), nickel(II) chloride, nickel(II) bromide, bis(triphenylphosphine)nickel(II) chloride or dichloro[1-1′-bis(diphenylphosphino)ferrocene]palladium(II). In addition, a free radical initiator may conveniently be added, for example an azo compound such as azo(bisisobutyronitrile).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, reductive amination of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green and P. G. M. Wuts, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Where any one of groups R^{1a′}, R^{1b′} or R^{1c′} is a precursor for a R^{1a}, R^{1b} or R^{1c} group respectively, it may converted into a compound of formula I by converting the precursor of any one of groups R^{1a}, R^{1b} or R^{1c} to the appropriate R^{1a}, R^{1b} or R^{1c} group using standard chemical techniques that are well known to those skilled in the art. Examples of possible R^{1a′}, R^{1b′} or R^{1c′} precursor groups (particularly R^{1b′} precursor groups) include hydroxy or alcohol-containing groups (e.g. —CH₂OH), aldehyde-containing groups (e.g. —CHO), carboxylic acid-containing groups (e.g. —(CH₂)₀₋₃—COOH) ester containing groups (e.g. —(CH₂)₀₋₃—COOR^{Z}, where R^{Z} is (1-4C)alkyl), activated ester containing groups (e.g. —(CH₂)₀₋₃—COOR^{Y}, where R^{Y} is a group such as, for example, pentafluorophenyl), amide containing groups (e.g. —CONH₂), acyl halide containing groups (e.g —(CH₂)₀₋₃—C(O)X, where X is a halogen such as for example chloride) ora group —CH₂—X where X is a reactive group as hereinbefore defined A person skilled in the art will appreciate how to select the most appropriate precursor group for conversion into the desired R^{1a}, R^{1b} or R^{1c} substituent groups.

For example, a suitable process (process (d)) for preparing compounds of structural formula (XIII):

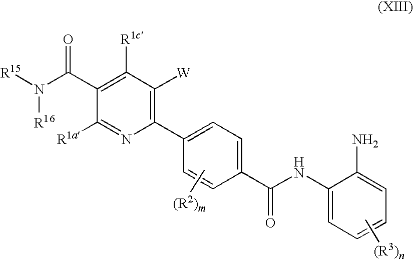

comprises the steps of:

reacting a compound of formula (G)

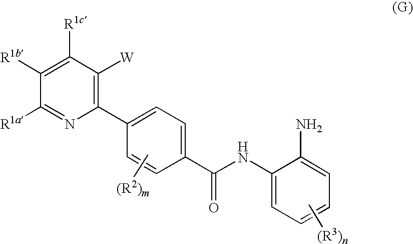

wherein the aniline may be suitably protected and R^{1b′} is a precursor group selected from —COOH, an acyl chloride (—C(O)Cl), an ester (—COOR^{z}) or an activated ester (—COOR^{y}) as herein before defined;

with a compound of formula (H)

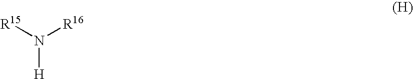

in a suitable solvent and in the presence of a suitable base; and thereafter, if necessary, removing any protecting groups that are present.

Where R^{1b′} is —COOH, the reaction is conveniently carried out in the presence of a suitable coupling agent. Examples of suitable coupling agents include HATU (O-(7-azabenzotriazol-1-yl)-N,N,N′,N′-tetramethyluronium hexafluorophosphate), EDCI (1-(3-dimethylaminopropyl)-3-ethylcarbodiimide), DCCI (1,3-dicyclohexylcarbodiimide), DMTMM (4-(4,6-dimethoxy-1,3,5-triazinyl-2-yl)-4-methylmorpholinium chloride), PYBOP (benzotriazole-1-yl-oxy-trispyrrolidinonophosphonium hexafluorophosphate) or CDI(1,1′-carbonyldiimidazole).

A suitable base for process (d) when R^{1b′} is —COOH is an organic amine base such as DIPEA (N,N-diisopropylethylamine), triethylamine or N-methylmorpholine.

The reaction can be carried out in solvents such as dichloromethane or DMF (N,N-dimethylformamide).

If R^{1b′} is a precursor group selected from an acyl chloride or an activated ester as hereinbefore defined, the reaction may be carried out in the presence of a suitable organic base in a solvent such as dichloromethane. If R^{1b′} is an ester precursor group (other than an activated ester) as herein before defined, then the reaction could be carried out in a solvent, such as an alcohol, without a base present.

If R^{1b′} is —COOH, it may be desirable to initially convert it into a more reactive group such as an acyl chloride or an activated ester prior to the reaction with compound (H). For example, the —COOH group can be converted into a more reactive —C(O)Cl group by reacting the compound of formula (G) with a suitable chlorinating agent such as, for example, thionyl chloride or oxalyl chloride. Alternatively, the —COOH group can be converted into an activated ester group (such as, for example, a pentafluorophenoxy ester by reacting the compound of formula (G) with pentafluorophenol in the presence of a suitable coupling reagent such as those defined previously for process (d), for example, HATU, EDCI, DCCI and in the presence of a suitable organic base such as DIPEA, triethylamine or N-methyl morpholine in dichloromethane).

A suitable process (process (e)) for preparing compounds of formula (XII):

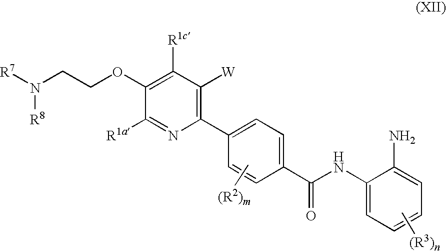

comprises the steps of:

reacting a compound of formula (J) wherein the aniline may be suitably protected

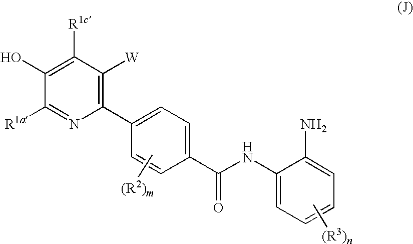

with a compound of formula (K)

R⁷R⁸N—CR^{a}R^{b}]ₐ—OH (K)

(wherein R⁷, R⁸, R^{a}, R^{b}, and integer a have any one of the definitions set out herein)

in a suitable solvent and under suitable dehydrating conditions; and thereafter, if necessary, removing any protecting groups.

Examples of a suitable solvent for use in process (e) include dichloromethane, THF or DMF or mixtures thereof.

Suitable dehydrating conditions for process (e) include, for example the use of a dialkyl azodicarboxylate, such as DEAD (diethyl azodicarboxylate), DIAD (diisopropyl azodicarboxylate), or DTAD (di-tert-butyl azodicarboxylate) in the presence of biphenyl phosphine

Alternatively, compounds of formula (J) can also be converted to compounds of formula (XII) by process (process (f)) comprising the reaction of a compound of formula (J) with a compound of formula (L) in the presence of a suitable base

R⁷R⁸N—[CR^{a}R^{b}]ₐ—X (L)

wherein X is a reactive group as hereinbefore defined and R⁷, R⁸, R^{a}, R^{b}, and integer a have any one of the definitions set out hereinbefore for the compounds of formula I; and thereafter, if necessary, removing any protecting groups.

A suitable base for process (f) would, for example, be an organic base such as triethylamine, DIPEA, N-methylmorpholine or an alkaline metal carbonate or hydroxide such as sodium carbonate, potassium carbonate, calcium carbonate, sodium hydroxide or potassium hydroxide or an alkali metal hydride such as sodium hydride or a metal alkoxide such as sodium ethoxide.

A further aspect of the current invention provides a process (process (g)) for preparing a compound of formula (J) above, said process comprising the steps of:

the reaction, in the presence of a suitable base and suitable catalyst, of a compound of the formula (M), wherein X is a reactive group as hereinbefore defined and the hydroxyl function may be suitably protected.

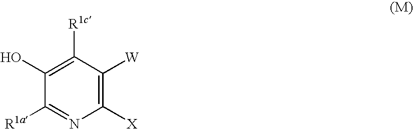

with a compound of formula (N), wherein L¹ and L² are ligands as hereinbefore defined;

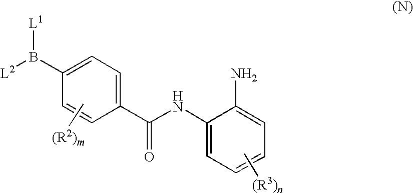

and thereafter if necessary, removing any protecting groups.

A suitable choice of base and catalyst for process (g) are as hereinbefore defined for processes (a) and (b).

In addition, an example of a suitable process (process (h)) for preparing a compound of the formula (X)

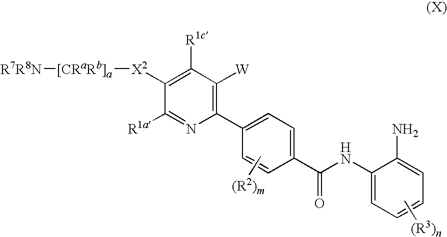

wherein X² is a direct bond, integer a is 1 or 2, and R⁷, R⁸, R^{a}, and R^{b} have any one of the definitions set out above;
comprises the steps of:

reacting a compound of formula (O), in the presence of a suitable base, and wherein X is a reactive group as hereinbefore defined and the aniline may be protected,

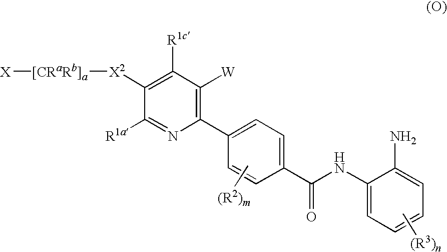

with a compound of formula (P)

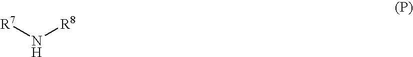

and thereafter, if necessary, removing any protecting groups.

A suitable base for use in process (h) is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal hydride, for example sodium hydride, an alkaline earth metal hydrogencarbonate such as sodium hydrogencarbonate, or a metal alkoxide such as sodium ethoxide.

A suitable process (process (i)) for preparing compound (Q) (i.e a compound of the formula (O) above where W is fluoro, X² is a direct bond, integer a is 1 and R^{a} and R^{b} are both hydrogen)

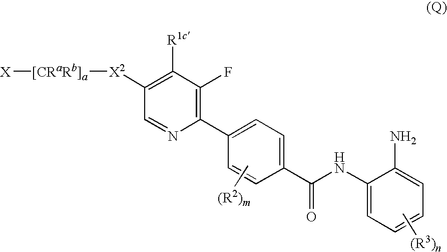

comprises the steps of:

(i) reaction of a compound of formula (R) wherein R^{1b′} is —COOH or an ester (—COOR^{Z}, where R^{Z} is (1-4C)alkyl) and the aniline may be suitably protected

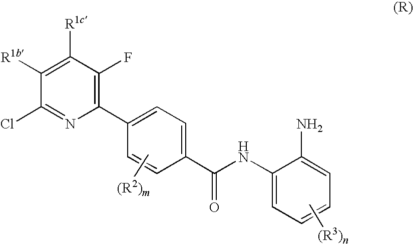

with a suitable reducing agent to produce a compound of formula (S);

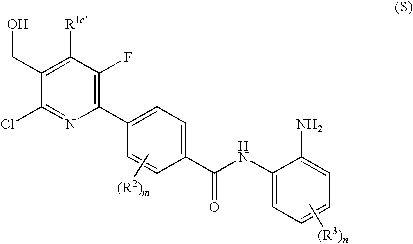

ii) dechlorinating the compound of formula (S) made in step (i) above under suitable conditions to produce a compound of formula (T)

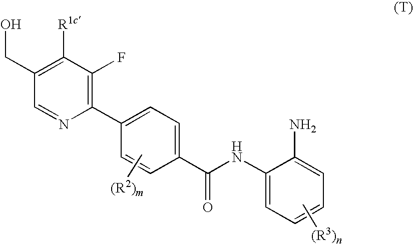

and thereafter in any suitable order or combination

iii) converting the compound of formula (T) into a compound of formula (Q) using convenient methods known to one skilled in the art; and/or

iv) if necessary, removing any protecting groups.

For example, the compound of formula (T) may be converted into a compound of formula (Q) in step (iii) of process (h) above by the reaction, in the presence of a suitable base, such as for example triethylamine, of a compound of formula (T) with methanesulfonyl chloride to produce a compound of formula (Q), where X² is a direct bond, integer a is 1, R^{a} and R^{b} are both hydrogen and reactive group X is a methanesulfonyloxy. A suitable reducing agent for use in process (i) step (i), includes, for example, an inorganic borohydride salt such as, sodium borohydride, sodium triacetoxyborohydride or sodium cyanoborohydride; or any aluminium hydride containing compound such as, for example, lithium aluminium hydride or diisobutylaluminim hydride.

Suitable dechlorinating conditions for use in process (i) step (ii) include, for example, the use of hydrogenolysis in the presence of a suitable catalyst and, if deemed necessary, a base, such as reacting the compound in the presence of 10% palladium on activated charcoal and triethylamine, under an atmosphere of hydrogen.

Alternatively, a compound of formula (XI) (being a compound of formula X above, wherein X² is a direct bond, integer a is 1, R^{a} and R^{b} are both hydrogen and R⁷ and R⁸ have any one of the definitions set out hereinbefore) may be prepared by a process (process (j))

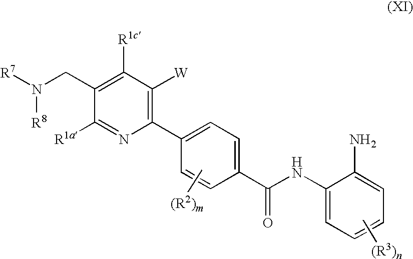

which comprises the reaction, in the presence of a suitable reducing agent and a suitable acid, of a compound of formula (U), wherein the aniline may be protected and R^{1b′} is a precursor for the R⁷R⁸N—CH₂— group in the compound of formula (XI) above, said precursor having the formula —CHO(formyl):

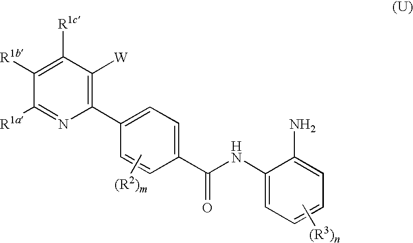

with a compound of formula (P);

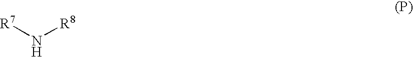

A suitable reducing agent for process (j) includes, for example, an inorganic borohydride salt such as, sodium borohydride, sodium triacetoxyborohydride or sodium cyanoborohydride.

A suitable acid for process (j), includes a Bronsted acid such as, for example formic acid, acetic acid, trifluoroacetic acid, hydrochloric acid, sulphuric acid, paratoluene sulfonic acid or camphor sulfonic acid; or a Lewis acid of formula MX_{z}, wherein M is a metal, X is a reactive group as hereindefined and z is in the range of 1-6 and the value of z will depend on the metal M. Typical examples of suitable Lewis acids include boron trifluoride, scandium(III) trifluoromethanesulfonate, tin(VI) chloride, titanium(IV) isopropoxide or zinc(II) chloride.

Examples of suitable solvents include methanol, acetic acid and dichloromethane or mixtures thereof.

Another aspect of the present invention provides a particular process (process (k)) for preparing the intermediate compound (U) above, wherein R^{1b′} is —CHO(formyl), said process comprising the steps of:

(i) the reaction, in the presence of a suitable base and suitable catalyst, of a compound of the formula (V), wherein X is a reactive group as hereinbefore defined and the hydroxyl function may be suitably protected.

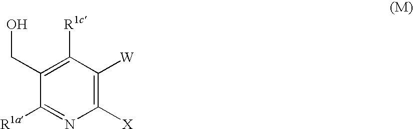

with a compound of formula (N), wherein L¹ and L² are ligands as hereinbefore defined;

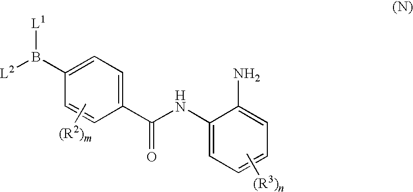

to produce a compound of formula (W);

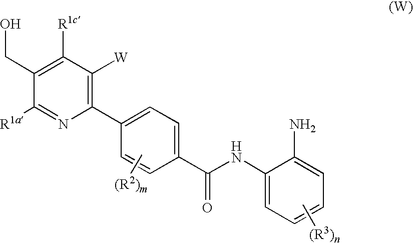

and thereafter in any suitable order or combination:
i) the reaction of a compound of formula (W) produced in step (i) under suitable oxidation conditions and/or
ii) if necessary, removing any protecting groups.

A suitable choice of base and catalyst for process (k), step (i) are as hereinbefore defined for processes (a) and (b).

A suitable choice of oxidation conditions/reagents for process (k), step (ii), includes the use of, for example, manganese dioxide, Dess-Martin periodinane (1,1,1-triacetoxy-1,1-dihydro-1,2-benziodoxol-3(1h)-one), pyridinium chlorochromate, chromium trioxide, oxalyl chloride and dimethyl sulfoxide (Swern oxidation) and TPAP (tetrapropylammonium erruthenate).

It will appreciated that in providing a process (process (k)) for the preparation of a compound of formula (U), a process for the preparation of a compound of formula (W) is also provided and that a compound of formula (W) is a direct precursor to a compound of formula (O) wherein X² is a direct bond, integer a is 1, R^{a} and R^{b} are both hydrogen and X is a reactive group as hereinbefore defined.

For example a compound of formula (O) where X² is a direct bond, integer a is 1, R^{a} and R^{b} are both hydrogen and X is methanesulfonyloxy (CH₃SO₂O—) may be prepared by the reaction of a compound of formula (W) with methanesulfonyl chloride in the presence of triethylamine in a suitable solvent, such as, for example tetrahydrofuran.

### Biological Assays

The following assays can be used to measure the effects of the compounds of the present invention as HDAC inhibitors, as inhibitors in vitro of recombinant human HDAC1 produced in Hi5 insect cells, and as inducers in vitro & in vivo of Histone H3 acetylation in whole cells and tumours. They also assess the ability of such compounds to inhibit proliferation of human tumour cells.
(a) In Vitro Enzyme Assay of Recombinant HDAC1

HDAC inhibitors were screened against recombinant human HDAC1 produced in Hi5 insect cells. The enzyme was cloned with a FLAG tag at the C-terminal of the gene and affinity purified using Anti-FLAG M2 agarose from SIGMA (A2220).

The deacetylase assays were carried out in a 50 μl reaction. HDAC1 (75 ng of enzyme) diluted in 15 μl of reaction buffer (25 mM TrisHC1 (pH 8), 137 mM NaCl, 2.7 mM KCl, 1 mM MgCl₂) was mixed with either buffer alone (10 μl) or buffer containing compound (10 μl for 30 minutes at ambient temperature. 25 μM acetylated histone H4 peptide (KI 174 Biomol) diluted in 25 μl of buffer was then added to the reaction and incubated for one hour at ambient temperature. The reaction was stopped by addition of an equal volume (50 μl) Fluor de Lys developer (Biomol) containing Trichostatin A at 2 μM. The reaction was allowed to develop for 30 minutes at ambient temperature and then fluorescence measured at an excitation wavelength of 360 nM and an emission wavelength of 465 nM. IC₅₀ values for HDAC enzyme inhibitors were determined by performing dose response curves with individual compounds and determining the concentration of inhibitor producing fifty percent decrease in the maximal signal (diluent control).
(b) In Vitro Assay of Inhibition of Proliferation in Whole Cells

Inhibition of proliferation in whole cells was assayed using the Promega cell titer 96 aqueous proliferation assay (Promega #G5421). HCT116 cells were seeded in 96 well plates at 1×10³ cells/well, and allowed to adhere overnight. They were treated with inhibitors for 72 hours. 20 μl of the tetrazolium dye MTS was added to each well and the plates were reincubated for 3 hours. Absorbance was then measured on a 96 well plate reader at 490 nM. The IC₅₀ values for HDAC inhibitors were determined by performing dose response curves with individual compounds and determining the concentration of inhibitor producing fifty percent decrease in maximal signal (diluent control).
(c) In Vitro Enzyme Assay of Histone Deacetylase Activity in Whole Cells

Histone H3 acetylation in whole cells was measured using immunohistochemistry and analysis using the Cellomics arrayscan. A549 or HCT116 cells were seeded in 96 well plates at 1×10⁴ cells/well, and allowed to adhere overnight. They were treated with inhibitors for 24 hours and then fixed in 1.8% formaldehyde in tris buffered saline (TBS) for one hour. Cells were permeabilized with ice-cold methanol for 5 minutes, rinsed in TBS and then blocked in TBS 3% low-fat dried milk for 90 minutes. Cells were then incubated with polyclonal antibodies specific for the acetylated histone H3 (Upstate #06-599) diluted 1 in 500 in TBS 3% milk for one hour. Cells were rinsed three times in TBS and then incubated with fluorescein conjugated secondary antibodies (Molecular Probes #A11008) & Hoechst 333542 (1 μg/ml) (Molecular Probes H3570) in TBS plus 1% Bovine serum albumin (Sigma #B6917) for one hour. Unbound antibody was removed by three rinses with TBS and after the final rinse 100 μl of TBS was added to the cells and the plates sealed and analysed using the Cellomics arrayscan.

EC₅₀ values for HDAC inhibitors were determined by performing dose response curves with individual compounds and then determining the concentration of inhibitor producing fifty percent of the maximal signal (reference compound control—Trichostatin A (Sigma)).

Although the pharmacological properties of the compounds of the formula (I) vary with structural change as expected, in general activity possessed by compounds of the Formula I, may be demonstrated at the following concentrations or doses in one or more of the above tests (a)-(b):

Test (a): IC₅₀ in the range, for example, <0.060 μM;

Test (b): IC₅₀ in the range, for example, <0.75 μM.

The following table discloses various biological data for a representative selection of compounds of the present invention. Comparative test data is also provided for N-(2-aminophenyl)-4-pyridin-2-ylbenzamide (Comparator).

| Example | | |
|---|---|---|
| (Compound No.) | Test (a) IC₅₀ (μM) | Test (b) IC₅₀ (μM) |
| 1 | 0.009 | 0.160 |
| 2 (8) | 0.016 | 0.4 |
| 5 (1) | 0.009 | 0.22 |
| Comparator | 0.089 | 2.33 |

According to a further aspect of the invention there is provided a pharmaceutical composition which comprises a compound of the formula (I), or a pharmaceutically acceptable salt or pro-drug thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

We have found that the compounds defined in the present invention, or a pharmaceutically acceptable salt thereof, are effective cell cycle inhibitors (anti-cell proliferation agents), which property is believed to arise from their HDAC inhibitory properties. We also believe that the compounds of the present invention may be involved in the inhibition of angiogenesis, activation of apoptosis and differentiation. Accordingly the compounds of the present invention are expected to be useful in the treatment of diseases or medical conditions mediated alone or in part by HDAC enzymes, i.e. the compounds may be used to produce a HDAC inhibitory effect in a warm-blooded animal in need of such treatment. Thus, the compounds of the present invention provide a method for treating the proliferation of malignant cells characterised by inhibition of HDAC enzymes, i.e. the compounds may be used to produce an anti-proliferative effect mediated alone or in part by the inhibition of HDACs.

According to one aspect of the present invention there is provided a compound of the formula (I), or a pharmaceutically acceptable salt or pro-drug thereof, as defined hereinbefore for use in a method of treatment of the human or animal body by therapy.

Thus according to a further aspect of the invention there is provided a compound of the formula (I), or a pharmaceutically acceptable salt or pro-drug thereof, as defined hereinbefore for use as a medicament.

According to a further aspect of the invention there is provided the use of a compound of the formula (I), or a pharmaceutically acceptable salt or pro-drug thereof, as defined hereinbefore in the manufacture of a medicament for use in the production of a HDAC inhibitory effect in a warm-blooded animal such as man.

According to a further feature of this aspect of the invention there is provided a method for producing a HDAC inhibitory effect in a warm-blooded animal, such as man, in need of such treatment which comprises administering to said animal an effective amount of a compound of the formula (I), or a pharmaceutically acceptable salt or pro-drug thereof, as defined hereinbefore.

According to a further aspect of the invention there is provided the use of a compound of the formula (I), or a pharmaceutically acceptable salt or pro-drug thereof, as defined hereinbefore in the manufacture of a medicament for use in the production of a cell cycle inhibitory (anti-cell-proliferation) effect in a warm-blooded animal such as man.

According to a further feature of this aspect of the invention there is provided a method for producing a cell cycle inhibitory (anti-cell-proliferation) effect in a warm-blooded animal, such as man, in need of such treatment which comprises administering to said animal an effective amount of a compound of the formula (I), or a pharmaceutically acceptable salt or pro-drug thereof, as defined hereinbefore.

According to an additional feature of this aspect of the invention there is provided a method of treating cancer in a waiin-blooded animal, such as man, in need of such treatment which comprises administering to said animal an effective amount of a compound of the formula (I), or a pharmaceutically acceptable salt or pro-drug thereof, as defined hereinbefore.

According to a further feature of the invention there is provided a compound of the formula (I), or a pharmaceutically acceptable salt or pro-drug thereof, as defined hereinbefore in the manufacture of a medicament for use in the treatment of cancer.

According to an additional feature of this aspect of the invention there is provided a compound of the formula (I), or a pharmaceutically acceptable salt or pro-drug thereof, or a pharmaceutically acceptable salt thereof, as defined hereinbefore, for use in the treatment of cancer.

In a further aspect of the present invention there is provided the use of a compound of the formula (I), or a pharmaceutically acceptable salt or pro-drug thereof, as defined hereinbefore, in the manufacture of a medicament for use in lung cancer, colorectal cancer, breast cancer, prostate cancer, lymphoma and/or leukaemia.

In a further aspect of the present invention there is provided a method of treating lung cancer, colorectal cancer, breast cancer, prostate cancer, lymphoma or leukaemia, in a warm-blooded animal, such as man, in need of such treatment which comprises administering to said animal an effective amount of a compound of the formula (I), or a pharmaceutically acceptable salt or pro-drug thereof, as defined hereinbefore.

Cancers that are amenable to treatment with the present invention include oesophageal cancer, myeloma, hepatocellular, pancreatic and cervical cancer, Ewings tumour, neuroblastoma, kaposis sarcoma, ovarian cancer, breast cancer, colorectal cancer, prostate cancer, bladder cancer, melanoma, lung cancer [including non small cell lung cancer (NSCLC) and small cell lung cancer (SCLC)], gastric cancer, head and neck cancer, brain cancer, renal cancer, lymphoma and leukaemia.

There is further provided is a compound of the formula (I), or a pharmaceutically acceptable salt or pro-drug thereof, as defined hereinbefore, for use in a method of treating inflammatory diseases, autoimmune diseases and allergic/atopic diseases.

In particular a compound of the formula (I), or a pharmaceutically acceptable salt thereof, as defined hereinbefore, is provided for use in a method of treating inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis), multiple sclerosis, atherosclerosis, spondyloarthropathies (ankylosing spondylitis, psoriatic arthritis, arthritis connected to ulcerative colitis), AIDS-related neuropathies, systemic lupus erythematosus, asthma, chronic obstructive lung diseases, bronchitis, pleuritis, adult respiratory distress syndrome, sepsis, and acute and chronic hepatitis (either viral, bacterial or toxic).

Further provided is a compound of the formula (I), or a pharmaceutically acceptable salt or pro-drug thereof, as defined hereinbefore, for use as a medicament in the treatment of inflammatory diseases, autoimmune diseases and allergic/atopic diseases in a warm-blooded animal such as man.

In particular a compound of the formula (I), or a pharmaceutically acceptable salt or pro-drug thereof, as defined hereinbefore, is provided for use as a medicament in the treatment of inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis), multiple sclerosis, atherosclerosis, spondyloarthropathies (ankylosing spondylitis, psoriatic arthritis, arthritis connected to ulcerative colitis), AIDS-related neuropathies, systemic lupus erythematosus, asthma, chronic obstructive lung diseases, bronchitis, pleuritis, adult respiratory distress syndrome, sepsis, and acute and chronic hepatitis (either viral, bacterial or toxic).

Further provided is the use of a compound of the formula (I), or a pharmaceutically acceptable salt thereof, as defined hereinbefore, in the manufacture of a medicament for use in the treatment of inflammatory diseases, autoimmune diseases and allergic/atopic diseases in a warm-blooded animal such as man.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The HDAC inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyl]otoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) Agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;

(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO 99/02166, WO00/40529, WO 00141669, WO01/92224, WO02/04434 and WO02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;

(x) Cell cycle inhibitors including for example CDK inhibitions (eg flavopiridol) and other inhibitors of cell cycle checkpoints (eg checkpoint kinase); inhibitors of aurora kinase and other kinases involved in mitosis and cytokinesis regulation (eg mitotic kinesins); and other histone deacetylase inhibitors; and

(xi) differentiation agents (for example retinoic acid and vitamin D).

According to this aspect of the invention there is provided a pharmaceutical composition comprising a compound of the formula (I) as defined hereinbefore and an additional anti-tumour substance as defined hereinbefore for the conjoint treatment of cancer.

In addition to their use in therapeutic medicine, the compounds of formula (I) and their pharmaceutically acceptable salts thereof, are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

The invention will now be illustrated in the following Examples in which, generally:

(i) operations were carried out at ambient temperature, i.e. in the range 17 to 25° C. and under an atmosphere of an inert gas such as argon unless otherwise stated;

(ii) evaporations were carried out by rotary evaporation in vacuo and work-up procedures were carried out after removal of residual solids by filtration;

(iii) column chromatography (by the flash procedure) and medium pressure liquid chromatography (MPLC) were performed on Merck Kieselgel silica (Art. 9385) or Merck Lichroprep RP-18 (Art. 9303) reversed-phase silica obtained from E. Merck, Darmstadt, Germany or using proprietory pre-packed normal phase silica catridges, for example Redisep™ disposable chromatography cartridges obtained from Presearch Ltd., Hitchin, UK, or high pressure liquid chromatography (HPLC) was performed on C18 reverse phase silica, for example on a Dynamax C-18 60 Å preparative reversed-phase column;

(iv) yields, where present, are not necessarily the maximum attainable;

(v) in general, the structures of the end-products of the Formula (I) were confirmed by nuclear magnetic resonance (NMR) and/or mass spectral techniques; fast-atom bombardment (FAB) mass spectral data were obtained using a Platform spectrometer and, where appropriate, either positive ion data or negative ion data were collected; NMR chemical shift values were measured on the delta scale [proton magnetic resonance spectra were determined using a Jeol JNM EX 400 spectrometer operating at a field strength of 400 MHz, Varian Gemini 2000 spectrometer operating at a field strength of 300 MHz or a Bruker AM300 spectrometer operating at a field strength of 300 MHz;

(vi) intermediates were not generally fully characterised and purity was assessed by thin layer chromatographic, HPLC, infra-red (IR) and/or NMR analysis;

(vii) melting points are uncorrected and were determined using a Mettler SP62 automatic melting point apparatus or an oil-bath apparatus; melting points for the end-products of the formula (I) were determined after crystallisation from a conventional organic solvent such as ethanol, methanol, acetone, ether or hexane, alone or in admixture;

(viii) the following abbreviations have been used:
DMF N,N-dimethylformamide
DCM dichloromethane
DME 1,2-dimethoxyethane
DMSO dimethylsulphoxide
THF tetrahydrofuran
HATU O-(7-Azabenzotriazol-1-yl)-N,N,N′,N′-tetramethyluronium hexafluorophosphate
EDCI 1-(3-Dimethylaminopropyl)-3-ethylcarbodiimide
DCCI 1,3-Dicyclohexylcarbodiimide
PYBOP Benzotriazole-1-yl-oxy-trispyrrolidinonophosphonium hexafluorophosphate
DEAD Diethyl azodicarboxylate
DIAD Diisopropyl azodicarboxylate
DTAD Di-tert-butyl azodicarboxylate
DIPEA N,N-Diisopropylethylamine
MeOH Methanol
TFA Trifluoroacetic acid

### EXAMPLE 1

### N-(2-Aminophenyl)-4-{5-[(butylamino)methyl]-3-chloropyridin-2-yl}benzamide

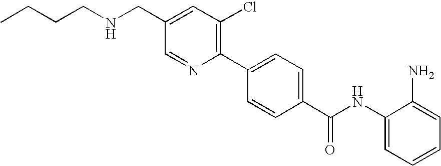

Titanium (IV) isopropoxide (397 μl, 379 mg, 1.33 mmol) was added to a stirred solution of tert-butyl (2-{[4-(3-chloro-5-formylpyridin-2-yl)benzoyl]amino}phenyl)carbamate (see Method 1 below, 301 mg, 0.667 mmol) and n-butylamine (73 mg, 1.00 mmol) in DCM (8 ml) at room temperature. The solution was stirred at ambient temperature for 1 hour, then sodium borohydride (101 mg, 2.67 mmol) and methanol (1 ml) were added. The mixture was stirred at ambient temperature for 18 hours then diluted with DCM (20 ml) and aqueous sodium bicarbonate solution (30 ml). The mixture was stirred for 5 minutes then filtered through a coarse filter cup. The aqueous layer was extracted with further DCM (2×50 ml), then the combined organics were washed with water (150 ml). The organic extracts were concentrated then chromatographed eluting with 0-0.5% 2N ammonia in methanol with ethylacetate to yield a white solid.

The white solid was dissolved in methanol (3 ml), and a 4M solution of hydrogen chloride in 1,4-dioxan (10 ml, 40 mmol) was added, then the solution stirred at ambient temperature for 18 hours. The solvent was evaporated, methanol (5 ml) added and the resulting solution was absorbed onto an SCX-2 column. This column was washed with methanol (2 column volumes) and the product eluted with a 2M solution of ammonia in methanol (2 column volumes) to give a foam. This was re-precipitated by stirring in diethyl ether (20 ml) to yield the title compound as a white solid (147 mg, 54%); NMR Spectrum: (DMSO-d₆) 0.90 (t, 3H), 1.37 (m, 2H), 1.49 (m, 2H), 2.63 (t, 2H), 3.87 (s, 2H), 4.71 (br s, 2H), 6.63 (m, 1H), 6.82 (m, 1H), 6.98 (m, 1H), 7.27 (m, 1H), 7.80 (d, 2H), 7.96 (m, 1H), 8.07 (d, 2H), 8.60 (m, 1M), 9.44 (br s, 1H); Mass Spectrum: M+H⁺ 409.

### EXAMPLE 2

Using an analogous procedure to that described in Example 1, the appropriate amine starting material was reacted to give the compounds described in Table 1.

TABLE 1

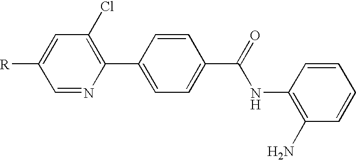

| Note | R | Analytical Data |
|---|---|---|
| 1 | CH₃—NH—CH₂— | NMR Spectrum: (DMSO-d₆) 2.47 (s, 3H), 3.77 (s, |
| | | 2H), 4.72 (br s, 2H), 6.63 (m, 1H), 6.82 (m, 1H), |
| | | 6.98 (m, 1H), 7.27 (m, 1H), 7.80 (d, 2H), 7.94 (m, |
| | | 1H), 8.07 (d, 2H), 8.58 (m, 1H), 9.44 (br s, 1H); |
| | | Mass Spectrum: M + H⁺ 367. |
| 2 | CH₃—CH₂—NH—CH₂— | NMR Spectrum: (DMSO-d₆) 1.07 (t, 3H), 2.62 (q, |
| | | 2H), 3.80 (s, 2H), 4.71 (br s, 2H), 6.63 (m, 1H), |
| | | 6.82 (m, 1H), 6.98 (m, 1H), 7.27 (m, 1H), 7.80 (d, |
| | | 2H), 7.94 (m, 1H), 8.07 (d, 2H), 8.58 (m, 1H), 9.44 |
| | | (br s, 1H); Mass Spectrum: M + H⁺ 381. |
| 3 | CH₃—CH₂—CH₂—NH—CH₂— | NMR Spectrum: (DMSO-d₆) 0.92 (t, 3H), 1.48 (m, |
| | | 2H), 2.56 (t, 2H), 3.80 (s, 2H), 4.71 (br s, 2H), 6.63 |
| | | (m, 1H), 6.82 (m, 1H), 6.98 (m, 1H), 7.27 (m, 1H), |
| | | 7.80 (d, 2H), 7.94 (m, 1H), 8.07 (d, 2H), 8.58 (m, |
| | | 1H), 9.44 (br s, 1H); Mass Spectrum: M + H⁺ 395. |
| 4 | CH₃—CH(CH₃)—NH—CH₂— | NMR Spectrum: (DMSO-d₆) 1.06 (d, 6H), 2.82 (m, |
| | | 1H), 3.83 (s, 2H), 4.71 (br s, 2H), 6.63 (m, 1H), |
| | | 6.82 (m, 1H), 6.98 (m, 1H), 7.27 (m, 1H), 7.80 (d, |
| | | 2H), 7.96 (m, 1H), 8.07 (d, 2H), 8.59 (m, 1H), 9.44 |
| | | (br s, 1H); Mass Spectrum: M + H⁺ 395. |
| 5 | 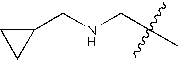 | NMR Spectrum: (DMSO-d₆) 0.13 (2H, m), 0.42 (m, 2H), 0.93 (m, 1H), 2.49 (m, 2H), 3.85 (s, 2H), 4.71 (br s, 2H), 6.63 (m, 1H), 6.82 (m, 1H), 6.98 (m, 1H), 7.27 (m, 1H), 7.80 (d, 2H), 7.96 (m, 1H), 8.07 (d, 2H), 8.59 (m, 1H), 9.44 (br s, 1H); Mass Spectrum: M + H⁺ 407. |
| 6 | 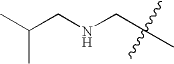 | NMR Spectrum: (DMSO-d₆) 0.91 (m, 6H), 1.72 (m, 1H), 2.40 (m, 2H), 3.71 (s, 2H), 4.71 (br s, 2H), 6.63 (m, 1H), 6.82 (m, 1H), 6.98 (m, 1H), 7.27 (m, 1H), 7.80 (d, 2H), 7.96 (m, 1H), 8.07 (d, 2H), 8.60 (m, 1H), 9.44 (br s, 1H); Mass Spectrum: M + H⁺ 409. |
| 7 | 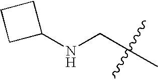 | NMR Spectrum: (DMSO-d₆) 1.70 (m, 4H, 2.12 (m, 2H), 3.36 (m, 1H), 3.74 (s, 2H), 4.71 (br s, 2H), 6.63 (m, 1H), 6.82 (m, 1H), 6.98 (m, 1H), 7.27 (m, 1H), 7.80 (d, 2H), 7.93 (m, 1H), 8.07 (d, 2H), 8.57 (m, 1H), 9.44 (br s, 1H); Mass Spectrum: M + H⁺ 407. |
| 8 | 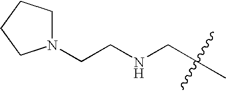 | NMR Spectrum: (DMSO-d₆) 1.74 (m, 4H), 2.57 (m, 4H), 2.66 (m, 2H), 2.73 (m, 2H), 3.86 (s, 2H), 4.72 (br s, 2H), 6.65 (m, 1H), 6.84 (m, 1H), 7.00 (m, 1H), 7.29 (m, 1H), 7.82 (d, 2H), 7.98 (m, 1H), 8.10 (d, 2H), 8.61 (m, 1H), 9.45 (br s, 1H); Mass Spectrum: M + H⁺ 450. |
| 9 | 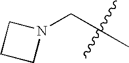 | NMR Spectrum: (DMSO-d₆ 373 K) 2.05 (m, 2H), 3.24 (m, 4H), 3.63 (s, 2H), 4.70 (br s, 2H), 6.63 (m, 1H), 6.82 (m, 1H), 6.98 (m, 1H), 7.27 (m, 1H), 7.79 (d, 2H), 7.86 (m, 1H), 8.07 (d, 2H), 8.52 (m, 1H), 9.43 (br s, 1H); Mass Spectrum: M + H⁺ 393. |

### EXAMPLE 3

### N-(2-Aminophenyl)-4-(3-chloropyridin-2-yl)benzamide

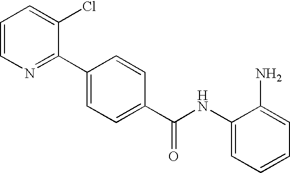

tert-Butyl (2-{[4-(3-chloropyridin-2-yl)benzoyl]amino}phenyl)carbamate (see Method 3 below, 1.60 g, 3.78 mmol) was dissolved in methanol (10 ml), and a 4M solution of hydrogen chloride in 1,4-dioxan (10 ml, 40 mmol) added then stirred at ambient temperature for 2 hours. The solvent was evaporated, methanol (5 ml) added and the resulting solution absorbed onto an SCX-2 column, washed with methanol (2 column volumes) and the product eluted with a 2M solution of ammonia in methanol (2 column volumes) to give a foam. This was re-precipitated by stirring in diethyl ether (20 ml) to give the product as a white solid (1.16 g, 95%); NMR Spectrum: (DMSO-d₆ 373K) 4.70 (br s, 2H), 6.63 (m, 1H), 6.82 (m, 1H), 6.97 (m, 1H), 7.28 (m, 1H), 7.44 (m, 1H), 7.80 (d, 2H), 8.02 (m, 1H), 8.08 (d, 2H), 8.66 (d, 1H), 9.46 (br s, 1H); Mass Spectrum: M+H⁺ 324.

### EXAMPLE 4

### N-(2-aminophenyl)-4-[3-chloro-5-(N-2-[dimethylamino]ethyl-N-methyl-carbamoyl)-pyridin-2-yl]benzamide (alternative name: 6-(4-{[(2-Aminophenyl)amino]carbonyl}phenyl)-5-chloro-N-[2-(dimethylamino)ethyl]-N-methylnicotinamide)

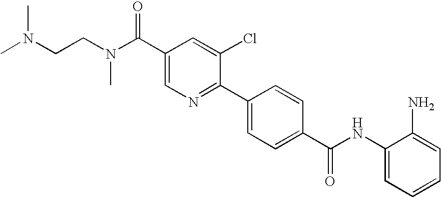

HATU (0.43 g, 1.12 mmol) was added to a solution 6-{4-({2-[(tert-butoxycarbonyl)amino]phenyl}amino)carbonyl]phenyl}-5-chloronicotinic acid (see Method 4, 0.35 g, 0.75 mmol), DIPEA (0.46 ml, 2.64 mmol) and N,N,N′-trimethylethylenediamine (0.097 g, 0.95 mmol) in dichloromethane (10 ml). After stirring for 3 hours the solution was washed with water (10 ml), and purified using flash column chromatography eluting with ethyl acetate followed by MeOH (10%) in dichloromethane (90%) to give a foam. This was dissolved in methanol (10 ml), and a solution of 4M hydrogen chloride in 1,4-dioxan (10 ml, 40 mmol) added then the solution stirred at ambient temperature for 2 hours. The solvent was evaporated, methanol (5 ml) added and the resulting solution absorbed onto an SCX-2 column, washed with methanol (2 column volumes) and the product eluted with a 2M solution of ammonia in methanol (2 column volumes) to give a foam. This was re-precipitated by stirring in diethyl ether (20 ml) to give the product as a white solid (112 mg, 36%); NMR Spectrum: (DMSO-d₆ 373K) 2.20 (s, 6H), 2.53 (m, 2H), 3.02 (s, 3H), 3.49 (m, 2H), 4.71 (br s, 2H), 6.64 (m, 1H), 6.82 (m, 1H), 6.98 (m, 1H), 7.27 (m, 1H), 7.84 (m, 2H), 8.05 (d, 1H), 8.10 (m, 2H), 8.65 (d, 1H), 9.47 (br s, 1H); Mass Spectrum: M+H⁺ 452

### EXAMPLE 5

Using an analogous procedure to that described in Example 4, amine starting material was reacted to give the compounds described in Table 2.

TABLE 2

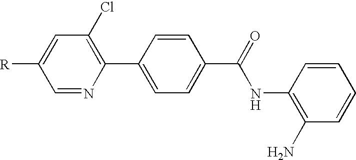

| Note | R | Analytical Data |
|---|---|---|
| 1 | 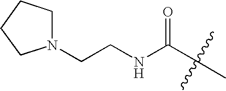 | NMR Spectrum: (DMSO-d₆ 373 K) 1.72 (m, 4H), 2.58 (m, 4H), 2.68 (t, 2H), 3.46 (m, 2H), 4.71 (br s, 2H), 6.64 (m, 1H), 6.82 (m, 1H), 6.98 (m, 1H), 7.27 (m, 1H), 7.84 (d, 2H), 8.10 (d, 2H), 8.37 (d, 1H), 8.45 (br s, 1H), 9.02 (d, 1H), 9.47 (br s, 1H); Mass Spectrum: M + H⁺ 464. |
| 2 | 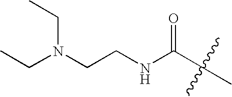 | NMR Spectrum: (DMSO-d₆ 373 K) 1.02 (t, 6H), 2.60 (m, 4H), 2.68 (m, 2H), 3.42 (m, 2H), 4.70 (br s, 2H), 6.64 (m, 1H), 6.82 (m, 1H), 6.98 (m, 1H), 7.27 (m, 1H), 7.86 (d, 2H), 8.10 (d, 2H), 8.38 (m, 1H), 8.42 (br s, 1H), 9.03 (m, 1H), 9.48 (br s, 1H); Mass Spectrum: M + H⁺ 466. |

### EXAMPLE 6

### N-(2-Aminophenyl)-4-(3-bromopyridin-2-yl)benzamide

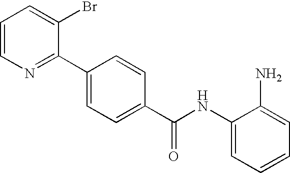

tert-butyl (2-{[4-(3-bromopyridin-2-yl)benzoyl]amino}phenyl)carbamate (see Method 5 below, 0.380 g, 0.8 mmol) was dissolved in methanol (10 ml), and a solution of hydrogen chloride in 1,4-dioxan (4M, 10 ml, 40 mmol) was added, and the solution stirred at ambient temperature for 2 hours. The solvent was evaporated, methanol (5 ml) added and the resulting solution absorbed onto an SCX-2 column, washed with methanol (2 column volumes) and the product eluted with a 2M solution of ammonia in methanol (2 column volumes) to give a foam. This was re-precipitated by stirring in diethyl ether (20 ml) to give the product as a white solid (0.210, 70%); NMR Spectrum: (DMSO-d₆ 373K) 4.70 (br s, 2H), 6.63 (m, 1H), 6.82 (m, 1H), 6.98 (m, 1H), 7.27 (m, 1H), 7.36 (m, 1H), 7.75 (d, 2H), 8.07 (d, 2H), 8.18 (m, 1H), 8.78 (m, 1H), 9.46 (br s, 1H); Mass Spectrum: M+H⁺370.

### EXAMPLE 7

### N-(2-Aminophenyl)-4-[3-chloro-5-(hydroxymethyl)pyridin-2-yl]benzamide

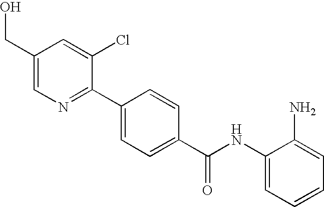

tert-Butyl [2-({4-[3-chloro-5-(hydroxymethyl)pyridin-2-yl]benzoyl}amino)phenyl]carbamate (see Method 2 below, 0.28 g, 0.62 mmol) was dissolved in methanol (10 ml), and a 4M solution of hydrogen chloride in 1,4-dioxan (10 ml, 40 mmol) was added, and the solution stirred at ambient temperature for 2 hours. The solvent was evaporated, methanol (5 ml) added and the resulting solution absorbed onto an SCX-2 column, washed with methanol (2 column volumes) and the product eluted with a 2M solution of ammonia in methanol (2 column volumes) to give a foam. This was re-precipitated by stirring in diethyl ether (20 ml) to give the product as a white solid (132 mg, 95%); NMR Spectrum: (DMSO-d₆) 4.62 (s, 2H), 4.94 (br s, 2H), 5.48 (br s, 1H), 6.61 (m, 1H), 6.79 (m, 1H), 6.98 (m, 1H), 7.20 (m, 1H), 7.80 (d, 2H), 7.96 (m, 1H), 8.07 (d, 2H), 8.60 (m, 1H), 9.73 (br s, 1H); Mass Spectrum: M+H⁺ 354.

### EXAMPLE 8

### N-(2-Aminophenyl)-4-[3-chloro-5-(pyrrolidin-1-ylmethyl)pyridin-2-yl]benzamide

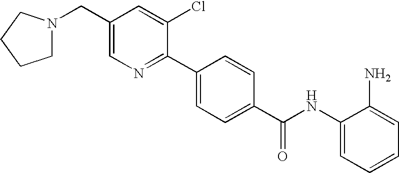

tert-Butyl (2-{[4-(3-chloro-5-formylpyridin-2-yl)benzoyl]amino}phenyl)carbamate (see Method 1 below, 0.30 g, 0.66 mmol) and pyrrolidine (0.058 mg, 0.82 mmol) were dissolved in dichloromethane (10 ml). Sodium triacetoxyborohydride (0.15 g, 0.718 mmol) was added, and the mixture stirred for 3 hours before being washed with water (10 ml). The organic residues were separated and purified using flash column chromatography eluting with ethyl acetate, followed by MeOH (5%) in dichloromethane (95%) to give tert-butyl [2-({4-[3-chloro-5-(pyrrolidin-1-ylmethyl)pyridin-2-yl]benzoyl}amino)phenyl]carbamate as a pale yellow oil. This was dissolved in methanol (10 ml), and a 4 M solution of hydrogen chloride in 1,4-dioxan (10 ml, 40 mmol) added, and the solution stirred at ambient temperature for 2 hours. The solvent was evaporated, methanol (5 ml) added and the resulting solution absorbed onto an SCX-2 column, washed with methanol (2 column volumes) and the product eluted with a 2M solution of ammonia in methanol (2 column volumes) to give a foam. This was re-precipitated by stirring in diethyl ether (20 ml) to give the product as a white solid (0.115 mg, 43%); NMR Spectrum: (DMSO-d₆ 373K) 1.78 (m, 4H), 2.58 (m, 4H), 3.74 (s, 2H), 4.72 (br s, 2H), 6.65 (m, 1H), 6.84 (m, 1H), 7.00 (m, 1H), 7.29 (m, 1H), 7.83 (m, 2H), 7.93 (m, 1H), 8.09 (m, 2H), 8.60 (m, 1H), 9.46 (br s, 1H); Mass Spectrum: M+H⁺ 407.

### EXAMPLE 9

Using an analogous procedure to that described in Example 8, the appropriate amine starting material was reacted to give the compounds described in Table 3.

TABLE 3

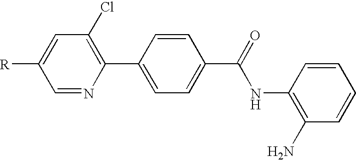

| Note | R | Analytical Data |
|---|---|---|
| 1 | 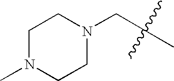 | NMR Spectrum: (DMSO-d₆ 373 K) 2.21 (s, 3H), 2.40 (m, 4H), 2.50 (m, 4H), 3.60 (s, 2H), 4.71 (br s, 2H), 6.63 (m, 1H), 6.82 (m, 1H), 6.98 (m, 1H), 7.27 (m, 1H), 7.81 (m, 2H), 7.90 (m, 1H), 8.07 (m, 2H), 8.57 (m, 1H), 9.43 (br s, 1H); Mass Spectrum: M + H⁺ 436. |
| 2 | 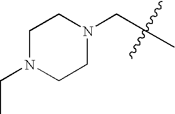 | NMR Spectrum: (DMSO-d₆ 373 K) 0.98 (t, 3H), 2.39 (q, 2H), 2.48 (m, 8H), 3.60 (s, 2H), 4.70 (br s, 2H), 6.63 (m, 1H), 6.82 (m, 1H), 6.98 (m, 1H), 7.27 (m, 1H), 7.81 (m, 2H), 7.90 (m, 1H), 8.07 (m, 2H), 8.57 (m, 1H), 9.44 (br s, 1H); Mass Spectrum: M + H⁺ 450. |
| 3 | 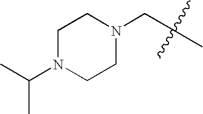 | NMR Spectrum: (DMSO-d₆ 373 K) 1.00 (d, 6H), 2.52 (m, 9H), 3.60 (s, 2H), 4.70 (br s, 2H), 6.63 (m, 1H), 6.82 (m, 1H), 6.98 (m, 1H), 7.27 (m, 1H), 7.81 (m, 2H), 7.90 (m, 1H), 8.07 (m, 2H), 8.57 (m, 1H), 9.43 (br s, 1H); Mass Spectrum: M + H⁺ 464. |
| 4 | 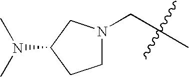 | NMR Spectrum: (DMSO-d₆ 373 K) 1.70 (m, 1H), 1.89 (m, 1H), 2.17 (s, 6H), 2.48 (m, 1H), 2.62 (m, 2H), 2.72 (m, 1H), 2.87 (m, 1H), 3.70 (dd, 2H), 4.70 (br s, 2H), 6.63 (m, 1H), 6.81 (m, 1H), 6.98 (m, 1H), 7.27 (m, 1H), 7.81 (m, 2H), 7.90 (m, 1H), 8.07 (m, 2H), 8.56 (m, 1H), 9.44 (br s, 1H); Mass Spectrum: M + H⁺ 450. |
| 5 | 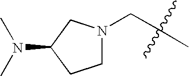 | NMR Spectrum: (DMSO-d₆ 373 K) 1.70 (m, 1H), 1.89(m, 1H), 2.17 (s, 6H), 2.48 (m, 1H), 2.62 (m, 2H), 2.72 (m, 1H), 2.87 (m, 1H), 3.70 (dd, 2H), 4.70 (br s, 2H), 6.63 (m, 1H), 6.81 (m, 1H), 6.98 (m, 1H), 7.27 (m, 1H), 7.81 (m, 2H), 7.90 (m, 1H), 8.07 (m, 2H), 8.56 (m, 1H), 9.44 (br s, 1H); Mass Spectrum: M + H⁺ 450. |

### EXAMPLE 10

### N-(2-Aminophenyl)-4-{3-chloro-5-[2-(dimethylamino)ethoxy]pyridin-2-yl}benzamide

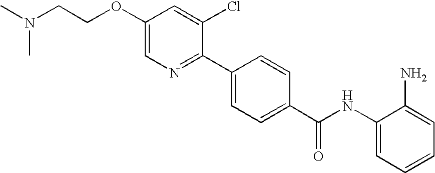

N-(2-Aminophenyl)-4-(3-chloro-5-hydroxypyridin-2-yl)benzamide (see Method 6 below; 0.30 g, 0.91 mmol), triphenyl phosphine (0.35 g, 1.33 mmol) and 2-dimethylaminoethanol (0.22 ml) were dissolved in DCM (10 ml) and DMF (0.40 ml). Diethyl azodicarboxylate (0.21 ml) was added and the solution stirred for 1 hour. This was purified using flash column chromatography eluting with MeOH (10%) in dichloromethane (90%) to give the product as a foam. This was re-precipitated by stirring in diethyl ether (20 ml) to give the product as a white solid (58 mg, 16%); NMR Spectrum: (DMSO-d₆ 373K) 2.30 (s, 6H), 2.72 (t, 2H), 4.26 (t, 2H), 4.70 (br s, 2H), 6.63 (m, 1H), 6.81 (m, 1H), 6.97 (m, 1H), 7.27 (m, 1H), 7.65 (d, 1H), 7.77 (m, 2H), 8.05 (m, 2H), 8.39 (d, 1H), 9.42 (br s, 1H); Mass Spectrum: M+H⁺ 411.

### EXAMPLE 11

Using an analogous procedure to that described in Example 10, the appropriate alcohol starting material was reacted to give the compounds described in Table 4.

TABLE 4

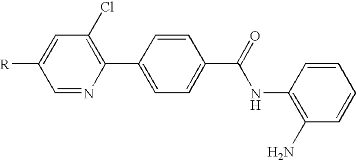

| Note | R | Analytical Data |
|---|---|---|
| 1 | 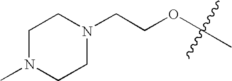 | NMR Spectrum: (DMSO-d₆) 2.15 (s, 3H), 2.32 (m, 6H), 2.71 (m, 4H), 4.25 (m, 2H), 4.91 (br s, 2H), 6.60 (m, 1H), 6.79 (m, 1H), 6.97 (m, 1H), 7.17 (m, 1H), 7.75 (m, 3H), 8.04 (m, 2H), 8.39 (m, 1H), 9.71 (br s, 1H); Mass Spectrum: M + H⁺ 466. |
| 2 | 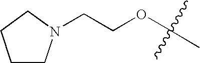 | NMR Spectrum: (DMSO-d₆ 373 K) 1.72 (m, 4H), 2.61 (m, 4H), 2.88 (t, 2H), 4.30 (t, 2H), 4.70 (br s, 2H), 6.63 (m, 1H), 6.81 (m, 1H), 6.97 (m, 1H), 7.27 (m, 1H), 7.65 (d, 1H), 7.77 (m, 2H), 8.05 (m, 2H), 8.39 (d, 1H), 9.42 (br s, 1H); Mass Spectrum: M + H⁺ 437. |
| 3 | 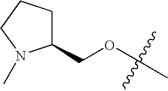 | NMR Spectrum: (DMSO-d₆ 373 K) 1.73 (m, 3H), 2.01 (m, 1H), 2.30 (m, 1H), 2.42 (s, 3H), 2.69 (m, 1H), 3.00 (m, 1H), 4.07 (m, 1H), 4.17 (m, 1H), 4.70 (br s, 2H), 6.63 (m, 1H), 6.81 (m, 1H), 6.97 (m, 1H), 7.27 (m, 1H), 7.65 (d, 1H), 7.77 (m, 2H), 8.05 (m, 2H), 8.39 (d, 1H), 9.42 (br s, 1H); Mass Spectrum: M + H⁺ 437. |

### EXAMPLE 12

### N-(2-Aminophenyl)-4-[5-(azetidin-1-ylmethyl)-3-fluoropyridin-2-yl]benzamide

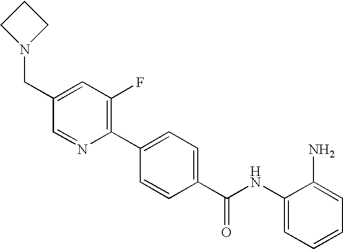

TFA (1 ml) was added to a solution of tert-butyl [2-({4-[5-(azetidin-1-ylmethyl)-3-fluoropyridin-2-yl]benzoyl}amino)phenyl]carbamate (see Method 7 below; 108 mg, 0.23 mmol) in dichloromethane (4 ml). The reaction mixture was stirred at ambient temperature for 30 minutes and then poured onto an SCX-2 cartridge (5 g). The cartridge was washed through with dichloromethane (5 column volumes) and MeOH (5 column volumes). Products were then eluted with a 2M solution of ammonia in methanol and the eluant evaporated to dryness and dried under high vacuum to afford the title compound (77 mg, 89%); NMR Spectrum: (DMSO-d₆) 2.03 (qn, 2H), 3.23 (m, 4H), 3.67 (s, 2H), 4.92 (s, 2H), 6.62 (m, 1H), 6.80 (d, 1H), 6.99 (m, 1H), 7.21 (d, 1H), 7.74 (d, 1H), 8.04 (d, 2H), 8.12 (d, 2H), 8.50 (s, 1H), 9.74 (s, 1H); Mass Spectrum: M+H⁺ 377.

### EXAMPLE 13

### N-(2-Aminophenyl)-4-{3-fluoro-5-[(4-isopropylpiperazin-1-yl)methyl]pyridin-2-yl}benzamide

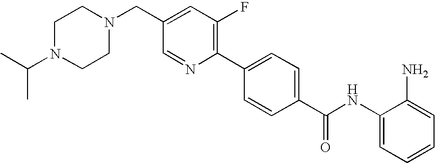

Using an analogous procedure to that described in Example 12, tert-butyl {2-[(4-{3-fluoro-5-[(4-isopropylpiperazin-1-yl)methyl]pyridin-2-yl}benzoyl)amino]phenyl}carbamate (see Method 8 below; 113 mg, 0.21 mmol) was reacted in dichloromethane (4 ml) and TFA (1 ml) to afford the title compound (93 mg, >99%); NMR Spectrum: (DMSO-d₆) 0.97 (s, 6H), 2.46 (m, 8H), 2.62 (m, 1H), 3.60 (s, 2H), 4.92 (s, 2H), 6.62 (m, 1H), 6.80 (d, 1H), 6.99 (m, 1H), 7.21 (d, 1H), 7.76 (d, 1H), 8.05 (d, 2H), 8.12 (d, 2H), 8.52 (s, 1H), 9.74 (s, 1H); Mass Spectrum: M+H⁺ 449.

### EXAMPLE 14

### N-(2-Aminophenyl)-4-{5-[(4-ethylpiperazin-1-yl)methyl]-3-fluoropyridin-2-yl}benzamide

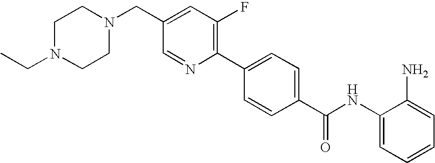

Using an analogous procedure to that described in method Example 12, tert-butyl {2-[(4-{5-[(4-ethylpiperazin-1-yl)methyl]-3-fluoropyridin-2-yl}benzoyl)amino]phenyl}carbamate (Method 9, 106 mg, 0.20 mmol) was reacted in dichloromethane (4 ml) and TFA (1 ml) to afford the title compound (83 mg, 96%); NMR Spectrum: (DMSO-d₆) 0.99 (t, 3H), 2.33 (m, 2H), 2.44 (m, 8H), 3.61 (s, 2H), 4.92 (s, 2H), 6.62 (m, 1H), 6.80 (d, 1H), 6.99 (m, 1H), 7.21 (d, 1H), 7.76 (d, 1H), 8.05 (d, 2H), 8.12 (d, 2H), 8.52 (s, 1H), 9.74 (s, 1H); Mass Spectrum: M+H⁺ 435.

### EXAMPLE 15

### N-(2-Aminophenyl)-4-(3-chloro-5-{[(3-methoxypropyl)amino]methyl}pyridin-2-yl)benzamide

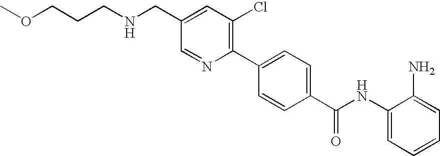

A solution of tert-butyl (2-{[4-(3-chloro-5-formylpyridin-2-yl)benzoyl]amino}phenyl)carbamate (Method 1; 181 mg, 0.40 mmol) in dichloromethane (4 ml) was added to 3-methoxypropylamine (67 mg, 0.75 mmol). The reaction mixture was stirred for 5 minutes then titanium (IV) isopropoxide (240 μl, 0.8 mmol) added. The reaction was allowed to stir at ambient temperature for 2 hours. Sodium borohydride (61 mg, 1.60 mmol), was added followed by methanol (0.4 ml) then stirred for a further 2 hours. Saturated aqueous sodium hydrogencarbonate solution (5 ml) was added, followed by water (5 ml) and dichloromethane (5 ml) the mixture stirred for 30 minutes. The organic phase was separated by filtration through a phase separating cartridge, and the aqueous extracted again with further aliquot of dichloromethane. The combined organic extracts were evaporated to dryness and the residue purified by flash chromatography on silica eluting with ethyl acetate followed by a rising gradient of methanol in ethyl acetate (0-20% v/v) to afford the protected product as an oil (147 mg). The residue was taken up in dichloromethane (3 ml) and treated with trifluoroacetic acid (1 ml) before stirring at ambient temperature for 22 hrs. The reaction mixture was diluted with dichloromethane and poured onto an IST SCX-2 cartridge (5 g). The cartridge was washed with dichloromethane (25 ml) and methanol (50 ml) before eluting the product with ammonia in methanol (2M solution, 50 ml). The ammoniacal fraction was evaporated to dryness and the resultant residue triturated with diethyl ether/isohexane to afford the title compound (84 mg, 49%); NMR Spectrum: (DMSO-d₆) δ 1.71 (m, 2H), 2.60 (m, 2H), 3.23 (s, 3H), 3.40 (t, 2H), 3.82 (s, 2H), 4.93 (s, 2H), 6.62 (m, 1H), 6.80 (m, 1H), 6.99 (m, 1H), 7.21 (m, 1H), 7.81 (d, 2H), 8.03 (m, 1H), 8.09 (d, 2H), 8.61 (m, 1H), 9.74 (s, 1H); Mass Spectrum: M+H⁺ (³⁵Cl) 425.

### EXAMPLE 16

Using an analogous procedure to that described in Example 15, tert-butyl (2-{[4-(3-chloro-5-formylpyridin-2-yl)benzoyl]amino}phenyl)carbamate (Method 1) was reacted with the appropriate amine starting material to give the compounds described in Table 5

TABLE 5

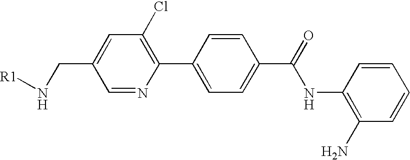

| Example | R¹ | Analytical Data | SM |
|---|---|---|---|
| 16.1 | 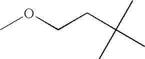 | NMR Spectrum: (DMSO-d₆); δ 2.69 (t, 2H), 3.26 (s, 3H), 3.43 (t, 2H), 3.83 (s, 2H), 4.93 (s, 2H), 6.62 (m, 1H), 6.80 (m, 1H), 6.99 (m, 1H), 7.21 (m, 1H), 7.81 (d, 2H), 8.02 (m, 1H), 8.09 (d, 2H), 8.60 (m, 1H), 9.74 (s, 1H); Mass Spectrum: M + H⁺ (³⁵Cl) 411. | |
| 16.2 | 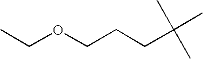 | NMR Spectrum: (DMSO-d₆) δ 1.10 (t, 3H), 1.68 (m, 2H), 2.58 (t, 2H), 3.41 (m, 4H), 3.80 (s, 2H), 4.93 (s, 2H), 6.62 (m, 1H), 6.80 (m, 1H), 6.99 (m, 1H), 7.21 (m, 1H), 7.81 (d, 2H), 8.01 (m, 1H), 8.09 (d, 2H), 8.60 (m, 1H), 9.74 (s, 1H); Mass Spectrum: M + H⁺ (³⁵Cl) 439. | |

### EXAMPLE 17

Using an analogous procedure to that described in Example 15, tert-butyl (2-{[4-(3-chloro-5-formylpyridin-2-yl)benzoyl]amino}phenyl)carbamate (Method 1) was reacted with the appropriate amine starting material to give the compounds described in Table 6

TABLE 6

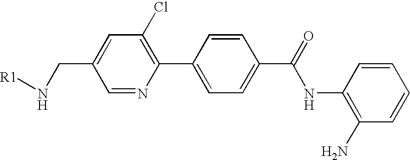

| Example | R¹ | Analytical Data | SM |
|---|---|---|---|
| 17.1 | 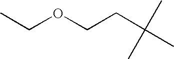 | NMR Spectrum: (DMSO-d₆) δ 1.12 (t, 3H) 2.70 (m, 2H), 3.45 (m, 4H), 3.84 (s, 2H), 4.93 (s, 2H), 6.62 (m, 1H), 6.80 (m, 1H), 6.99 (m, 1H), 7.21 (m, 1H), 7.81 (d, 2H), 8.03 (m, 1H), 8.09 (d, 2H), 8.61 (m, 1H), 9.74 (s, 1H); Mass Spectrum: M + H⁺ (³⁵Cl) 425. | |
| 17.2 | 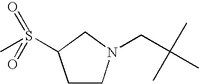 | NMR Spectrum: (CDCl₃) δ 2.25 (m, 2H), 2.65 (m, 1H), 2.77 (m, 1H), 2.82 (s, 3H), 2.99 (m, 2H), 3.55 (m, 1H), 3.66 (s, 2H), 3.80 (br s, 2H), 6.80 (m, 2H), 7.04 (m, 1H), 7.32 (d, 1H), 7.74 (s, 1H), 7.82 (m, 3H), 7.94 (d, 2H), 8.47 (s, 1H); Mass Spectrum: M + H⁺ (³⁵Cl) 485. | |

### EXAMPLE 18

### N-(2-Aminophenyl)-4-(3-chloro-5-{[4-(2-methoxyethyl)piperazin-1-yl]methyl}pyridin-2-yl)benzamide

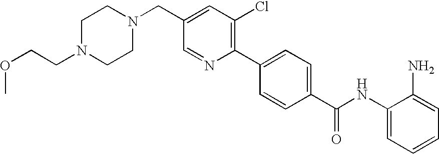

A solution of tert-butyl (2-{[4-(3-chloro-5-formylpyridin-2-yl)benzoyl]amino}phenyl)carbamate (Method 1; 181 mg, 0.40 mmol) in dichloromethane (4 ml) was added to 1-(2-methoxyethyl)piperazine (102 mg, 0.70 mmol). The reaction mixture was stirred for 5 minutes before addition of titanium (IV) isopropoxide (240 μl, 0.8 mmol). The reaction was allowed to stir at ambient temperature for 2 hours then sodium borohydride (61 mg, 1.60 mmol), added followed by methanol (0.4 ml). The reaction mixture was stirred for 2 hours then a further portion of sodium borohydride (61 mg, 1.60 mmol) added the reaction mixture was left to stir overnight (20 hours). Saturated aqueous sodium hydrogencarbonate solution (5 ml) was added, followed by water (5 ml) and dichloromethane (5 ml). The mixture was stirred for 30 minutes and the organic phase separated by filtration through an IST phase separating cartridge, and the aqueous extracted again with dichloromethane. The combined organic extracts were evaporated to dryness and the residue purified by flash chromatography on silica eluting with ethyl acetate followed by a rising gradient of methanol in ethyl acetate (0-20% v/v) to afford the protected product. This was taken up in dichloromethane (3 ml) and treated with trifluoroacetic acid (1 ml) then at ambient temperature for 22 hrs. The reaction mixture was diluted with dichloromethane and poured onto an SCX-2 cartridge (5 g). The cartridge was washed with dichloromethane (25 ml) and methanol (50 ml) before eluting products with a 2M solution of ammonia in methanol (50 ml). The ammoniacal fraction was evaporated to dryness and the resultant residue triturated with diethyl ether/isohexane to afford the title compound (78 mg, 41%); NMR Spectrum: (CDCl₃) δ 2.61 (m, 10H), 3.35 (s, 3H), 3.53 (t, 2H), 3.58 (s, 2H), 3.88 (s, 2H), 6.87 (m, 2H), 7.11 (m, 1H), 7.38 (d, 1H), 7.84 (d, 2H), 7.88 (s, 2H), 8.00 (d, 2H), 8.53 (s, 1H); Mass Spectrum: M+H⁺ (³⁵Cl) 480.

### EXAMPLE 19

### N-(2-Aminophenyl)-4-(3-chloro-5-{[(2-propoxyethyl)amino]methyl}pyridin-2-yl)benzamide

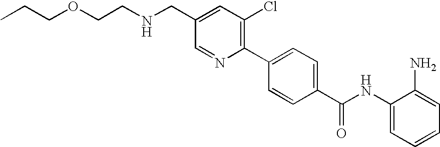

Diisopropylethylamine (230 μl, 1.32 mmol.) was added to a solution of tert-butyl [2-({4-[3-chloro-5-(hydroxymethyl)pyridin-2-yl]benzoyl}amino)phenyl]carbamate (Method 1; 200 mg, 0.44 mmol) in tetrahydrofuran (5 ml). The solution was cooled in an ice bath and for 5 minutes then methanesulfonyl chloride (50 μl, 0.65 mmol) added. The reaction mixture was allowed to stir for 90 minutes then added to a solution of 2-N-propoxyethylamine (414 mg, 4.00 mmol) in tetrahydrofuran (1.5 ml). The mixture was stirred at ambient temperature overnight (16.5 hours) and then heated to 50° C. for 2 hours, then heated to 65° C. for a further 3 hours. The cooled reaction mixture was partitioned between dichloromethane and water and the organic phase separated. The aqueous layer was re-extracted with more dichloromethane and the combined extracts were washed with brine, dried over magnesium sulphate, filtered and evaporated to dryness. The residue was purified by flash chromatography on silica, eluting with ethyl acetate, followed by a rising gradient of methanol in ethyl acetate (0-20% v/v). This afforded a straw-coloured foam (157 mg), which was taken up in dichloromethane (4 ml) and treated with triflouroacetic acid (1 ml). The mixture was stirred overnight, then diluted with dichloromethane and poured onto an SCX-2 cartridge (10 g). The cartridge was washed through with dichloromethane (50 ml) and methanol (100 ml) before eluting products with ammonia in methanol (2M solution, 100 ml). The ammoniacal fraction was evaporated to dryness and the resultant residue triturated under diethyl ether/isohexane to afford the title compound (95 mg, 49%); NMR Spectrum: (DMSO-d₆) δ 0.88 (t, 3H), 1.52 (m, 2H), 2.44 (s, 1H), 2.69 (t, 2H), 3.34 (t, 2H), 3.46 (t, 2H), 3.84 (s, 2H), 4.93 (s, 2H), 6.62 (m, 1H), 6.80 (m, 1H), 6.99 (m, 1H), 7.21 (m, 1H), 7.81 (d, 2H), 8.02 (m, 1H), 8.09 (d, 2H), 8.60 (m, 1H), 9.74 (s, 1H); Mass Spectrum: M+H⁺ (³⁵Cl) 439.

### Methods

### Method 1

### tert-Butyl (2-{[4-(3-chloro-5-formylpyridin-2-yl)benzoyl]amino}phenyl)carbamate

Dess-Martin periodinane (2.80 g, 6.61 mmol) was added to a stirred solution of tert-butyl [2-({4-[3-chloro-5-(hydroxymethyl)pyridin-2-yl]benzoyl}amino)phenyl]carbamate (see Method 2 below; 5.51 g, 5.54 mmol) in DCM (175 ml) at room temperature. The mixture was stirred at ambient temperature for 20 hours, then diluted with DCM (150 ml) and washed with aqueous 2N sodium hydroxide solution (4×100 ml), then saturated brine (100 ml). The organic extracts were dried over magnesium sulfate and treated with decolourising carbon then concentrated to yield the title compound as a pink solid (1,98 g, 80%); NMR Spectrum: (DMSO-d₆) 1.47 (s, 9H), 7.19 (m, 2H), 7.60 (m, 2H), 7.92 (d, 2H), 8.11 (d, 2H), 8.52 (m, 1H), 8.69 (br s, 1H), 9.15 (m, 1H), 9.94 (br s, 1H), 10.17 (s, 1H); Mass Spectrum: M+H⁺ 452.

### Method 2

### tert-Butyl [2-({4-[3-chloro-5-(hydroxymethyl)pyridin-2-yl]benzoyl}amino)phenyl]carbamate

1,1′-Bis(diphenylphosphino)ferrocenedichloropalladium (II) chloride (1.18 g, 1.45 mmol) was added to a mixture of N-(2-tert-butoxycarbonylaminophenyl)-4-(4,4,5,5-tetramethyl-1,3,2-dioxaborolan-2-Abenzamide (12.71 g, 29.0 mmol, prepared as described in International patent publication number WO 03/087057, Method 13, page 60), 5,6-dichloro-3-pyridinemethanol (5.15 g, 29.0 mmol) and saturated aqueous sodium hydrogen carbonate solution (65 ml) in 1,2-dimethoxyethane (130 ml). The mixture was heated at 60° C. for 5 hours, then cooled to room temperature and poured onto water (900 ml). The product was extracted with DCM (2×400 ml), dried (magnesium sulfate then decolourising carbon) and concentrated. The product was chromatographed (eluting with ethyl acetate/isohexane 65:35) to yield the title compound as a white solid (10.03 g, 76%); NMR Spectrum: (DMSO-d₆) 1.46 (s, 9H), 4.62 (d, 2H), 5.51 (t, 1H), 7.19 (m, 2H), 7.58 (d, 2H), 7.84 (d, 2H), 7.98 (m, 1H), 8.09 (d, 2H), 8.62 (m, 1H), 8.69 (br s, 1H), 9.92 (br s, 1H); Mass Spectrum: M+Na⁺ 476.

### Method 3

### tert-Butyl (2-{[4-(3-chloropyridin-2-yl)benzoyl]amino}phenyl)carbamate

N-(2-tert-Butoxycarbonylaminophenyl)-4-(4,4,5,5,tetramethyl-1,3,2,-dioxaborolan-2-yl) benzamide (2.96 g, 6.76 mmol, prepared as described in International patent publication number WO03/087057, Method 13, page 60), 2,3-dichloropyridine (1.00 g, 6.76 mmol), 1,1′-bis(diphenylphosphino)ferrocene-palladium(II)dichloride dichloromethane complex (276 mg, 0.338 mmol), 1,2-dimethoxyethane (30 ml) and a saturated aqueous solution of sodium hydrogen carbonate (15 ml) were stirred at 60° C. under an atmosphere of nitrogen for 7.5 hours. The mixture was allowed to cool before being partitioned between dichloromethane and water. The organics were separated, dried over sodium sulfate, filtered and evaporated. The crude product was purified by chromatography on silica eluting with 30% ethyl acetate in isohexane to afford the title compound as a white solid (1.87 g, 51%); NMR Spectrum: (DMSO-d₆) 1.47 (s, 9H), 7.20 (m, 2H), 7.50 (m, 1H), 7.60 (m, 2H), 7.86 (d, 2H), 8.10 (m, 3H), 8.69 (m, 2H), 9.92 (br s, 1H); Mass Spectrum: M+H⁺ 424.

### Method 4

### 6-{4-[({2-[(tert-Butoxycarbonyl)amino]phenyl}amino)carbonyl]phenyl}-5-chloronicotinic acid

Using an analogous procedure to that described in Method 3, the N-(2-tert-butoxycarbonylaminophenyl)-4-(4,4,5,5-tetramethyl-1,3,2-dioxaborolan-2-yl) benzamide starting material (17.10 g, 39 mmol) was reacted with 5,6-dichloronicotinic acid (7.50 g, 39 mmol) to give 6-{4-[({2-[(tert-butoxycarbonyl)amino]phenyl}amino)carbonyl]phenyl}-5-chloronicotinic acid (12.83 g, 70%) as a white solid. NMR Spectrum: (DMSO-d₆) 1.47 (s, 9H), 7.19 (m, 2H), 7.59 (m, 1H), 7.86 (d, 2H), 8.10 (d, 2H), 8.26 (m, 1H), 8.74 (m, 1H), 9.00 (m, 1H), 10.00 (br s, 1H); Mass Spectrum: M+H⁺ 468.

### Method 5

### tert-Butyl (2-{[4-(3-bromopyridin-2-yl)benzoyl]amino}phenyl)carbamate

Using an analogous procedure to that described in Method 3, the N-(2-tert-butoxycarbonylaminophenyl)-4-(4,4,5,5-tetramethyl-1,3,2-dioxaborolan-2-yl) benzamide starting material (1.79 g, 4.08 mmol) was reacted with 2,3-dibromopyridine (1.00 g, 4.2 mmol) to give tert-butyl (2-{[4-(3-bromopyridin-2-yl)benzoyl]amino}phenyl)carbamate (0.380 g, 20%) as a white solid. NMR Spectrum: (DMSO-d₆) 1.47 (s, 9H), 7.20(m, 2H), 7.40 (m, 1H), 7.59(m, 2H), 7.79 (m, 2H), 8.08 (m, 2H), 8.27 (m, 1H), 8.70 (m, 2H), 9.92 (br s, 1H); Mass Spectrum: M+H⁺ 470.

### Method 6

### N-(2-Aminophenyl)-4-(3-chloro-5-hydroxypyridin-2-yl)benzamide

Sodium methoxide (3.26 g, 60.4 mmol) was added to a solution of 5,6-dichloropyridin-3-ol (8.98 g, 54.7 mmol; CAS 110860-92-9 Reference: Koch, V; Schatterers, S; Synthesis (1990), 6, 499) in DMF (230 ml) at 0° C. After 15 minutes 2-methoxyethoxymethyl chloride was added dropwise, and the mixture allowed to stir at room temperature overnight. The DMF was removed under reduced pressure and the residue partitioned between diethyl ether (200 ml) and water (200 ml). The organic phase was washed with brine (200 ml), dried over magnesium sulphate and concentrated in vacuo to give 2,3-dichloro-5-[(2-methoxyethoxy)methoxy]pyridine (12.05 g) as an oil. Mass Spectrum: M+H⁺ 252.

Using an analogous procedure to that described in Method 3, the N-(2-tert-butoxycarbonylaminophenyl)-4-(4,4,5,5-tetramethyl-1,3,2-dioxaborolan-2-yl) benzamide (4.39 g, 10 mmol) starting material was reacted with 2,3-dichloro-5-[(2-methoxyethoxy)methoxy]pyridine (2.52 g, 10 mmol) to give tert-butyl {2-[(4-{3-chloro-5-[(2-methoxyethoxy)methoxy]pyridin-2-yl}benzoyl)amino]phenyl}carbamate (4.90 g, 94%) as a white solid. Mass Spectrum: M+H⁺ 528. tert-Butyl {2-[(4-{3-chloro-5-[(2-methoxyethoxy)methoxy]pyridin-2-yl}benzoyl)amino]phenyl}carbamate (4.50 g) was stirred in a solution of hydrogen chloride in 1,4-dioxan (4M, 50 ml, 200 mmol) for 2 hours. The solvent was evaporated, methanol (5 ml) added and the resulting solution absorbed onto an SCX-2 column, washed with methanol (2 column volumes) and the product eluted with a 2M solution of ammonia in methanol (2 column volumes) to give N-(2-aminophenyl)-4-(3-chloro-5-hydroxypyridin-2-yl)benzamide as a solid. NMR Spectrum: (DMSO-d₆ 373K) 3.20 (s, 1H), 4.70 (br s, 2H), 6.63 (m, 1H), 6.81 (m, 1H), 6.97 (m, 1H), 7.27 (m, 1H), 7.38 (d, 1H), 7.75 (m, 2H), 8.03 (m, 2H), 8.24 (d, 1H), 9.42 (br s, 1H); Mass Spectrum: M+H⁺ 340.

### Method 7

### tert-Butyl [2-({4-[5-(azetidin-1-ylmethyl)-3-fluoropyridin-2-yl]benzoyl}amino)phenyl]carbamate

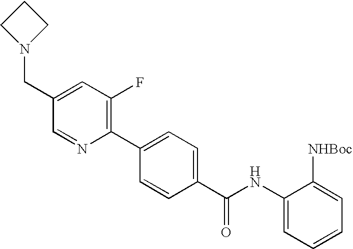

Methanesulfonyl chloride (90 μl, 1.16 mmol) and triethylamine (160 μl, 1.18 mmol) were added to a solution of tert-butyl [2-({4-[3-fluoro-5-(hydroxymethyl)pyridin-2-yl]benzoyl}amino)phenyl]carbamate (Method 10, 200 mg, 0.46 mmol) in dichloromethane (5 ml), at ambient temperature then stirred, under nitrogen, for 2 hours. Azetidine (200 μl, 2.97 mmol) was added and stirring continued for a further 18 hours. The reaction mixture was diluted with dichloromethane (20 ml) and washed with water and brine, dried over magnesium sulphate, filtered and evaporated to dryness. The residue was purified by flash chromatography, eluting with 10% methanol in dichloromethane, to afford the title compound (108 mg, 49%); Mass Spectrum: M+H⁺ 477.

### Method 8

### tert-Butyl {2-[(4-{3-fluoro-5-[(4-isopropylpiperazin-1-yl)methyl]pyridin-2-yl}benzoyl)amino]phenyl}carbamate

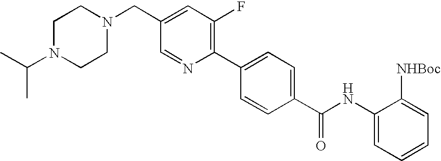

Using an analogous procedure to that described in Method 7, tert-butyl [2-({4-[3-fluoro-5-(hydroxymethyl)pyridin-2-yl]benzoyl}amino)phenyl]carbamate (Method 10, 160 mg, 0.37 mmol) was reacted with triethylamine (130 μl, 0.96 mmol), methanesulfonyl chloride (75 μl, 0.97 mmol) and 1-isopropylpiperazine (260 μl, 1.82 mmol) to afford the title compound (113 mg, 57%); Mass Spectrum: M+H⁺ 549.

### Method 9

### tert-Butyl {2-[(4-{5-[(4-ethylpiperazin-1-yl)methyl]-3-fluoropyridin-2- yl}benzoyl)amino]phenyl}carbamate

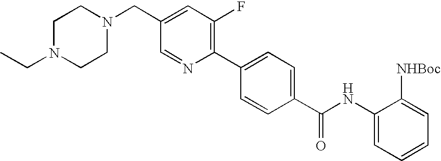

Using an analogous procedure to that described in Method 7, tert-butyl [2-({4-[3-fluoro-5-(hydroxymethyl)pyridin-2-yl]benzoyl}amino)phenyl]carbarnate (Method 10, 160 mg, 0.37 mmol) was reacted with triethylamine (130 μl, 0.96 mmol), methanesulfonyl chloride (75 μl, 0.97 mmol) and 1-ethlypiperazine (230 μl, 1.81 mmol) to afford the title compound (106 mg, 54%); Mass Spectrum: M+H⁺ 535.

### Method 10

### tert-Butyl [2-({4-[3-fluoro-5-(hydroxymethyl)pyridin-2-yl]benzoyl}amino)phenyl]carbamate

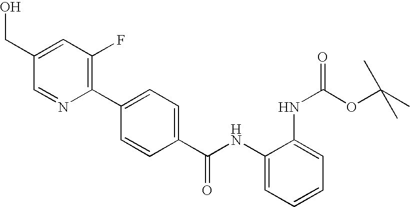

A 1.0 M solution of lithium aluminium hydride in tetrahydrofuran (8 ml, 8 mmol) was added dropwise to a solution of 6-{4-[({2-[(tert-butoxycarbonyl)amino]phenyl}amino)carbonyl]phenyl}-2-chloro-5-fluoronicotinic acid (Method 11, 1.7 g, 3.50 mmol) in tetrahydrofuran (80 ml), over 15 minutes at ambient temperature. Upon complete addition the reaction mixture was cooled in an ice bath and the reaction treated sequentially with water (300 μl), 15% aq. sodium hydroxide solution (300 μl) and water (1 ml). The reaction mixture was allowed to stir for 2 hours, allowing warming to room temperature. All solids were removed by filtration through celite, washing the filter cake with tetrahydrofuran and methanol. Evaporation of the filtrate to dryness gave an orange gum, which was re-dissolved in ethanol (100 ml) and treated with triethylamine (2 ml, 26.49 mmol) and 10% palladium on activated charcoal (580 mg). The mixture was placed under an atmosphere of hydrogen and stirred for 18 hours. The catalyst was removed by filtration through celite and the filtrate evaporated to dryness. The resultant residue was purified by flash chromatography, eluting with 0-10% methanol in dichloromethane to afford the title compound (322 mg, 21%); NMR Spectrum: (DMSO-d₆) 1.46 (s, 9H), 4.65 (m, 2H), 5.51 (br m, 1H), 7.20 (m, 2H), 7.58 (m, 2H), 7.77 (d, 1H), 8.10 (m, 4H), 8.56 (m, 1H), 8.68 (m, 1H), 9.92 (s, 1H); Mass Spectrum: M+H⁺ 438.

### Method 11

### 6-{4-[({2-(tert-Butoxycarbonyl)amino]phenyl}amino)carbonyl]phenyl}-2-chloro-5-fluoronicotinic acid

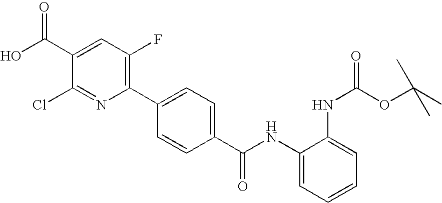

2,6-Dichloro-5-fluoropyridine-3-carboxylic acid (5.3 g, 25.2 mmol) and 1,1′-bis(diphenylphosphino)ferrocene]dichloropalladium(II) (0.82 g, 1.0 mmol) was added to a solution of N-(2-tert-butoxycarbonylaminophenyl)-4-(4,4,5,5-tetramethyl-1,3,2-dioxaborolan-2-yl) benzamide (prepared as described in International patent publication number WO03/087057 method 13, page 60, 10 g, 22.8 mmol) in 1,2-dimethoxyethane (200 ml). A saturated aqueous solution of sodium bicarbonate (100 ml) was then added carefully and the reaction mixture heated to 70° C. for 16 hours. Further 1,1′-bis(diphenylphosphino) ferrocene]dichloropalladium(II) (0.41 g, 0.5 mmol) was added and the reaction mixture heated at 85° C. for 18 hours. After cooling to room temperature the mixture was partitioned between ethyl acetate and water. The aqueous layer was separated and the organics washed with water. The combined aqueous washes were acidified to pH 2 by dropwise addition of concentrated hydrochloric acid and extracted with dichloromethane (×2). The combined extracts were washed with brine, dried over magnesium sulphate, filtered and evaporated to dryness. Trituration under diethyl ether gave a solid precipitate that was removed by filtration. Evaporation of the filtrate afforded the title compound (5.4 g, 49%); NMR Spectrum: (DMSO-d₆) 1.45 (s, 9H), 7.18 (m, 2H), 7.56 (m, 2H), 8.11 (m, 4H), 8.34 (d, 1H), 8.67 (s, 1H), 9.95 (s, 1H); Mass Spectrum: M+H⁺ 486.

1. A compound of formula (I) 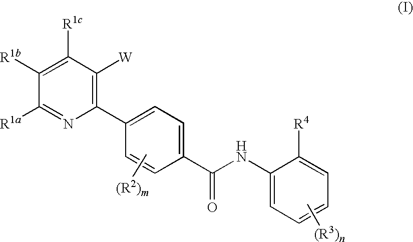
wherein:
R^{1a} is selected from hydrogen, amino, (1-3C)alkyl, N-(1-3C)alkylamino, N,N-di-(1-3C)alkylamino, or a group of the sub-formula II:
R⁵R⁶N—X¹—[CR^{a}R^{b}]_{q}— (II)
wherein:
q is 1, 2 or 3;
each R^{a} and R^{b} group present is independently selected from hydrogen, halo, hydroxy or (1-4C)alkyl;
X¹ is selected from a direct bond or —C(O)—; and
R⁵ and R⁶ are each independently selected from hydrogen or (1-3C)alkyl;
and wherein if R^{1a} is a N-(1-3C)alkylamino or N,N-di-(1-3C)alkylamino group, the (1-3C)alkyl moiety is optionally substituted by hydroxy or (1-2C)alkoxy;
R^{1b} is selected from:
(i) hydrogen, (1-6C)alkyl, halo(1-6C)alkyl, hydroxy(1-6C)alkyl, (3-6C)cycloalkyl, (3-6C)cycloalkyl(1-6C)alkyl, (1-6C)alkoxy, (1-6C)alkoxy(1-6C)alkyl, N-(1-6C)alkylsulphamoyl, N,N-di-[(1-6C)alkyl]sulphamoyl; or
(ii) a group of sub-formula III:
R⁷R⁸N—[CR^{a}R^{b}]ₐ—X²— (III)
wherein:
X² is selected from a direct bond, —O— or —C(O)—;
a is 0, 1, 2, 3 or 4;
R^{a} and R^{b} are as defined above;
R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IV:
R⁹R¹⁰N—[CR^{a}R^{b}]_{b}—X⁴— (IV)
wherein:
b is 1, 2 or 3;
R^{a} and R^{b} are as defined above;
X⁴ is a direct bond or —C(O)—;
R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by one or more groups selected from hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);
or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by one or more groups selected from hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S, or a group —[CH₂]_{f}—NR¹³R¹⁴ or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl); or
(iii) a group of the formula V:
R¹⁵R¹⁶N—X³—[CR^{a}R^{b}]_{c}— (V)
wherein:
c is 0, 1, 2 or 3;
R^{a} and R^{b} are as defined above;
X³ is —C(O)—;
R¹⁵ and R¹⁶ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula VI:
R¹⁷R¹⁸N—[CR^{a}R^{b}]_{d}— (VI)
wherein:
d is 1, 2 or 3;
R^{a} and R^{b} are as defined above;
R¹⁷ and R¹⁸ are independently selected from hydrogen, (1-6C)alkyl, (3-6C)cycloalkyl, (3-6C)cycloalkyl(1-6C)alkyl, (1-6C)alkoxy(1-6C)alkyl, or R¹⁷ and R¹⁸ are linked so that, together with the nitrogen atom to which they are attached, they form a 4-, 5-, 6- or 7-membered heterocyclic ring, said heterocyclic ring optionally comprising, in addition to the nitrogen atom to which R¹⁷ and R¹⁸ are attached, one or two further nitrogen atoms, and wherein the heterocyclic ring is optionally substituted by 1, 2 or 3, substituents selected from hydroxy, halo, (1-4C)alkyl, carbamoyl, oxo, or —[CH₂]_{g}—NR¹⁹R²⁰ (wherein g is 0, 1 or 2, and R¹⁹ and R²⁰ are independently selected from hydrogen, (1-6C)alkyl, (3-6C)cycloalkyl or (3-6C)cycloalkyl(1-6C)alkyl);
or R¹⁵ and R¹⁶ are linked so that, together with the nitrogen atom to which they are attached, they form a 4-, 5-, 6- or 7-membered heterocyclic ring, said heterocyclic ring optionally comprising, in addition to the nitrogen atom to which R¹⁵ and R¹⁶ are attached, one or two further nitrogen atoms and the heterocyclic ring is optionally substituted by 1, 2 or 3, substituents selected from hydroxy, halo, (1-4C)alkyl, carbamoyl, oxo, or —[CH₂]ₕ—NR²¹R²² (wherein h is 0, 1 or 2, and R²¹ and R²² are independently selected from hydrogen, (1-6C)alkyl, (3-6C)cycloalkyl or (3-6C)cycloalkyl(1-6C)alkyl);
(iv) a group of the sub-formula VII:
Q-Z—Y— (VII)
wherein:
Y is a direct bond or —[CR^{a}R^{b}]ₓ—, where x is 1 to 4 and R^{a} and R^{b} are as defined above;
Z is absent or selected from —O—, —S—, —SO—, —SO₂—, —NH—SO₂—,
—SO₂—NH— or —C(O)—; and
Q is a carbon-linked heterocyclyl or a heterocyclyl-(1-6C)alkyl group, said heterocyclyl or a heterocyclyl-(1-6C)alkyl group being optionally substituted on the heterocyclyl ring by one or more substituent groups (for example 1, 2 or 3), which may be the same or different, selected from halo, oxo, cyano, hydroxy, trifluoromethyl, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, (1-3C)alkoxy(1-3C)alkyl, (1-3C)alkoxycarbonyl, halo(1-3C)alkyl, N-[(1-3C)alkyl]amino, N,N-di-[(1-3C)alkyl]amino, N-[(1-3C)alkoxy(1-3C)alkyl]amino, N,N-di-[(1-3C)alkoxy(1-3C)alkyl]amino, N-[(1-3C)alkoxy(1-3C)alkyl]-N-[(1-3C)alkyl]amino, N-(1-3C)alkylcarbamoyl, N,N-di-[(1-3C)alkyl]carbamoyl, (1-3C)alkylthio, (1-3C)alkylsulphinyl, (1-3C)alkylsulphonyl, N-(1-3C)alkylsulphamoyl, N,N-di-[(1-3C)alkyl]sulphamoyl;
R^{1c} is selected from hydrogen, halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;
m is 0, 1, 2, 3 or 4;
R² is halo;
n is 0, 1, 2, 3 or 4;
R³ is selected from halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-Di(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;
R⁴ is amino or hydroxy; and
W is fluoro, chloro or bromo;
or a pharmaceutically acceptable salt or pro-drug thereof

2. A compound according to claim 1 wherein R^{1a} is hydrogen.

3. A compound according to claim 1 wherein R^{1b} is selected from hydrogen, or a group of sub-formula (III) as defined above.

4. A compound according to claim 1 wherein R^{1b} is a group of sub-formula (III).

5. A compound according to claim 4 wherein the group of sub-formula (III) is a group
R⁷R⁸N—[CR^{a}R^{b}]ₐ—X²— (III)
where R⁷, R⁸ and X² are as defined in claim 1, a is 1 and R^{a} and R^{b} are both hydrogen.

6. A compound according to claim 4 wherein X² is a direct bond.

7. A compound according to claim 4 wherein R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl.

8. A compound according to claim 7 wherein one of R⁷ or R⁸ is hydrogen or (1-6C)alkyl, and the other is (1-6C)alkyl or (1-6C)alkoxy(1-6C)alkyl.

9. A compound according to claim 4 wherein R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by one or more groups selected from hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl).

10. A compound according to claim 9 wherein R⁷ and R⁸ are linked so that they form a 4-, 5- or 6-membered heterocyclic ring which contains one, two or three nitrogen atoms, and wherein said heterocyclic ring is optionally substituted by one or more groups selected from hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl.

11. A compound according to claim 1 wherein R^{1c} is hydrogen.

12. A compound according to claim 1 wherein W is fluoro or chloro.

13. A compound according to claim 1 wherein R⁴ is amino.

14. A compound according to claim 1 wherein m is 0.

15. A compound according to claim 1 wherein n is 0.

16. A compound according to claim 1 which is selected from:
N-(2-aminophenyl)-4-(3-chloropyridin-2-yl)benzamide;
N-(2-aminophenyl)-4-[3-chloro-5-(N-2-[dimethylamino]ethyl-N-methyl-carbamoyl)-pyridin-2-yl]benzamide (alternative name: 6-(4-{[(2-aminophenyl)amino]carbonyl}phenyl)-5-chloro-N-[2-(dimethylamino)ethyl]-N-methylnicotinamide);
N-(2-aminophenyl)-4-[3-chloro-5-(N-2-[pyrrolidin-1-yl]ethyl-carbamoyl)-pyridin-2-yl]benzamide (alternative name: 6-(4-{[(2-aminophenyl)amino]carbonyl}phenyl)-5-chloro-N-(2-pyrrolidin-1-ylethyl)nicotinamide);
N-(2-aminophenyl)-4-(3-bromopyridin-2-yl)benzamide;
N-(2-aminophenyl)-4-{3-chloro-5-[(methylamino)methyl]pyridin-2-yl}benzamide;
N-(2-aminophenyl)-4-{3-chloro-5-[(ethylamino)methyl]pyridin-2-yl}benzamide;
N-(2-aminophenyl)-4-{3-chloro-5-[(propylamino)methyl]pyridin-2-yl}benzamide;
N-(2-aminophenyl)-4-{3-chloro-5-[(isopropylamino)methyl]pyridin-2-yl}benzamide;
N-(2-aminophenyl)-4-(3-chloro-5-{[(cyclopropylmethyl)amino]methyl}pyridin-2-yl)benzamide;
N-(2-aminophenyl)-4-[3-chloro-5-(N-2-[diethylamino]ethyl-carbamoyl)-pyridin-2-yl]benzamide (alternative name: 6-(4-{[(2-aminophenyl)amino]carbonyl}phenyl)-5-chloro-N-[2-(diethylamino)ethyl]nicotinamide;
N-(2-aminophenyl)-4-[3-chloro-5-(hydroxymethyl)pyridin-2-yl]benzamide;
N-(2-aminophenyl)-4-{3-chloro-5-[(4-methylpiperazin-1-yl)methyl]pyridin-2-yl}benzamide;
N-(2-aminophenyl)-4-{3-chloro-5-[(4-ethylpiperazin-1-yl)methyl]pyridin-2-yl}benzamide;
N-(2-aminophenyl)-4-{3-chloro-5-[(4-isopropylpiperazin-1-yl)methyl]pyridin-2-yl}benzamide;
N-(2-aminophenyl)-4-[3-chloro-5-(pyrrolidin-1-ylmethyl)pyridin-2-yl]benzamide;
N-(2-aminophenyl)-4-(3-chloro-5-{[(3S)-3-(dimethylamino)pyrrolidin-1-yl]methyl}pyridin-2-yl)benzamide;
N-(2-aminophenyl)-4-(3-chloro-5-{[(3R)-3-(dimethylamino)pyrrolidin-1-yl]methyl}pyridin-2-yl)benzamide;
N-(2-aminophenyl)-4-[5-(azetidin-1-ylmethyl)-3-chloropyridin-2-yl]benzamide;
N-(2-aminophenyl)-4-{5-[(butylamino)methyl]-3-chloropyridin-2-yl}benzamide;
N-(2-aminophenyl)-4-{3-chloro-5-[(isobutylamino)methyl]pyridin-2-yl}benzamide;
N-(2-aminophenyl)-4-{3-chloro-5-[(cyclobutylamino)methyl]pyridin-2-yl}benzamide;
N-(2-aminophenyl)-4-(3-chloro-5-{[(2-pyrrolidin-1-ylethyl)amino]methyl}pyridin-2-yl)benzamide;
N-(2-aminophenyl)-4-{3-chloro-5-[2-(dimethylamino)ethoxy]pyridin-2-yl}benzamide;
N-(2-aminophenyl)-4-{3-chloro-5-[2-(4-methylpiperazin-1-yl)ethoxy]pyridin-2-yl}benzamide;
N-(2-aminophenyl)-4-[3-chloro-5-(2-pyrrolidin-1-ylethoxy)pyridin-2-yl]benzamide;
N-(2-aminophenyl)-4-(3-chloro-5-{[(25)-1-methylpyrrolidin-2-yl]methoxy}pyridin-2-yl)benzamide;
N-(2-aminophenyl)-4-[5-(azetidin-1-ylmethyl)-3-fluoropyridin-2-yl]benzamide;
N-(2-aminophenyl)-4-{3-fluoro-5-[(4-isopropylpiperazin-1-yl)methyl]pyridin-2-yl}benzamide;
N-(2-aminophenyl)-4-{5-[(4-ethylpiperazin-1-yl)methyl]-3-fluoropyridin-2-yl}benzamide;
N-(2-aminophenyl)-4-(3-chloro-5-{[(3-methoxypropyl)amino]methyl}pyridin-2-yl)benzamide;
N-(2-aminophenyl)-4-(3-chloro-5-{[(2-methoxyethyl)amino]methyl}pyridin-2-yl)benzamide;
N-(2-aminophenyl)-4-(3-chloro-5-{[(3-ethoxypropyl)amino]methyl}pyridin-2-yl)benzamide;
N-(2-aminophenyl)-4-(3-chloro-5-{[(2-ethoxyethyl)amino]methyl}pyridin-2-yl)benzamide;
N-(2-aminophenyl)-4-(3-chloro-5-{[3-(methylsulfonyl)pyrrolidin-1-yl]methyl}pyridin-2-yl)benzamide;
N-(2-aminophenyl)-4-(3-chloro-5-{[4-(2-methoxyethyl)piperazin-1-yl]methyl}pyridin-2-yl)benzamide; and
N-(2-aminophenyl)-4-(3-chloro-5-{[(2-propoxyethyl)amino]methyl}pyridin-2-yl)benzamide.

17. A pharmaceutical composition which comprises a compound according to claim 1, or a pharmaceutically acceptable salt or pro-drug thereof, in association with a pharmaceutically-acceptable diluent or carrier.

18. A compound according to claim 1, or a pharmaceutically acceptable salt or pro-drug thereof, for use in a method of treatment of the human or animal body by therapy.

19. The use of a compound according claim 1 or a pharmaceutically acceptable salt or pro-drug thereof, in the manufacture of a medicament for use in the production of a HDAC inhibitory effect in a warm-blooded animal such as man.

20. The use of a compound according to claim 1 or a pharmaceutically acceptable salt or pro-drug thereof, in the manufacture of a medicament for use in the treatment of cancer.

21. The use of a compound according to claim 1 or a pharmaceutically acceptable salt or pro-drug thereof, in the manufacture of a medicament for use in the treatment of the treatment of inflammatory diseases, autoimmune diseases and allergic/atopic diseases in a warm-blooded animal.

22. A method for producing a HDAC inhibitory effect in a warm-blooded animal, such as man, in need of such treatment which comprises administering to said animal an effective amount of a compound of the formula (I) according to claim 1, or a pharmaceutically acceptable salt or pro-drug thereof.

23. A process for preparing a compound of formula (I) as defined in claim 1 or a pharmaceutically acceptable salt or pro-drug form thereof, said process comprising the steps of:
(a) the reaction of a compound of the formula (A) 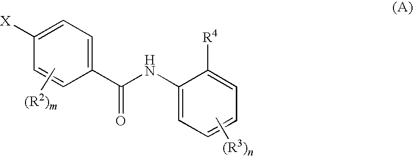
wherein R², R³, R⁴, m and n are as defined in claim 1 and X is a reactive group, with a compound of the formula (B) 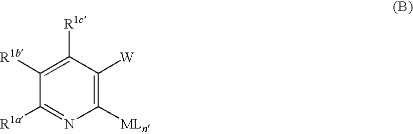
wherein
R^{1a′} is a group R^{1a} as hereinbefore defined or a precursor thereof,
R^{1b′} is a group R^{1b} as hereinbefore defined or a precursor thereof,
R^{1c′} is a group R^{1c} as hereinbefore defined or a precursor thereof,
M is a metal,
L is a ligand, and
integer n′ is 0 to 3;
and wherein if any one of said groups R^{1a′}, R^{1b′} or R^{1c′} is a precursor for a R^{1a}, R^{1b} or R^{1c} group respectively, then said process thereafter comprises a step of converting the compound formed by the reaction of a compound of the formula (A) with a compound of the formula (B) to a compound of formula (I) (by converting the precursor of any one of groups R^{1a}, R^{1b} or R^{1c} to the appropriate R^{1a}, R^{1b} or R^{1c} group); or
(b) The reaction of a compound of the formula (C) 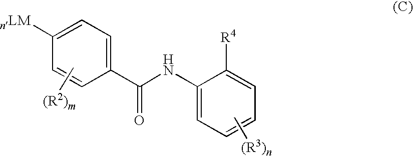
wherein R², R³, R⁴, m and n are as defined in claim 1 and M, L and integer n′ are as defined above, with a compound of the formula (D) 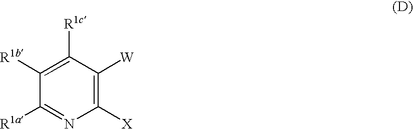
wherein W is as defined in claim 1, R^{1a′}, R^{1b′} and R^{1c′} are as defined above and X is a reactive group;
and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (C) with a compound of the formula (D) to a compound of formula (I) (by converting the precursor of any one of groups R^{1a}, R^{1b} or R^{1c} to the appropriate R^{1a}, R^{1b} or R^{1c} group); or
(c) an amide coupling reaction of a compound of the formula (E) 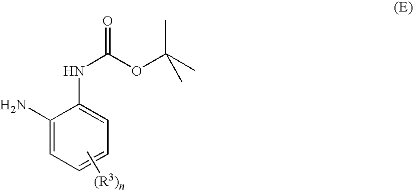
where R³ and n are as defined in claim 1, with a compound of the formula (F) 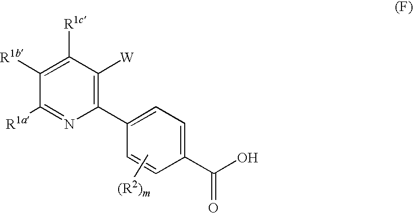
wherein W, R² and m are as defined in claim 1, R^{1a′}, R^{1b′} and R^{1c′} are as defined above, and wherein if any one of said groups R^{1a′}, R^{1b′} or R^{1c′} is a precursor for a R^{1a}, R^{1b} or R^{1c} group respectively, then said process comprises an additional step thereafter of converting the compound formed by the reaction of a compound of the formula (E) with a compound of the formula (F) to a compound of formula (I) (by converting the precursor of any one of groups R^{1a}, R^{1b} or R^{1c} to the appropriate R^{1a}, R^{1b} or R^{1c} group);
and thereafter if necessary:
i) converting a compound of the formula (I) into another compound of the formula (I); and/or
ii) removing any protecting groups.
